(19) United States
(12) Patent Application Publication
(10) Pub. No.: US 2014/0276768 A1
(43) Pub. Date: Sep. 18, 2014

(54) ELECTROSURGICAL SYSTEMS

(71) Applicant: Ellman International, Inc., Hickville, NY (US)

(72) Inventors: Albert M. Juergens, Boylston, MA (US); Frank D'Amelio, Los Olivos, CA (US)

(73) Assignee: Ellman International, Inc., Hickville, NY (US)

(21) Appl. No.: 14/205,021

(22) Filed: Mar. 11, 2014

Related U.S. Application Data
(60) Provisional application No. 61/786,038, filed on Mar. 14, 2013.

(51) Int. Cl. A61B 18/12

(52) U.S. Cl. CPC A61B 18/1233
USPC 606/34

(57) ABSTRACT

An electrosurgical instrument can selectively provide electrosurgical energy to an energizable electrode. The instrument can include an interface configured to issue one or more waveform command signals corresponding to a selected therapeutic result; waveform generation circuitry configured to deliver in response to the one or more wave form command signals, a corresponding one or more waveforms selected from a first, a second, and a third waveform, each of the first, second and third waveforms being different from the other of the first, second and third waveforms. The instrument can include a variable output voltage supply configured to supply a selected electrical current to a corresponding output, and an output driver circuitry configured to combine at least one or more of the waveforms with the respective high-voltage electrical current to generate an electrosurgical current suitable for achieving the therapeutic result when applied to a treatment site by the energizeable electrode.

### RELATED APPLICATIONS

This application claims the benefit of and priority to U.S. Patent Application No. 61/786,038, filed Mar. 14, 2013, the contents of which are hereby incorporated by reference as if recited in full herein for all purposes.

### BACKGROUND

The innovations and related subject matter disclosed herein (collectively referred to as the “disclosure”) generally pertain to electrosurgical systems, for example electrosurgical generators and related electrical circuitry and methods. More particularly but not exclusively, the innovations relate to electrosurgical systems configured to selectively deliver one or more therapeutic forms of electrical energy to a patient. As but one example, an innovative electrosurgical system can be configurable by (A) a user; (B) a “smart instrument”; or (C) a combination thereof, to deliver a selected therapeutic form of electrical energy to the patient. In general, a selected therapeutic form of electrical energy can comprise a steady-state or a time-varying combination of voltage, current, carrier frequency and wave form, each of which in turn can be steady state or time varying.

Electrosurgical instruments have been widely used in the aesthetic, medical, dental, and veterinarian fields. Such instruments can allow a user to precisely cut tissue using electrosurgical currents delivered to the tissue by a handpiece having a needle, a ball, or a loop electrode, e.g., in a monopolar operating mode, or to conveniently coagulate tissue using, for example, a forceps, e.g., in a bipolar operating mode.

In a typical surgical setting, a surgeon might first use a unipolar (or monopolar) handpiece to perform a desired cutting procedure and subsequently use a bipolar forceps to coagulate blood vessels. U.S. Pat. No. 5,954,686, incorporated herein by reference in its entirety, for all purposes, discloses an approach for maintaining a sterile field while still allowing the surgeon to connect and disconnect a variety of handpieces.

Known electrosurgical instruments typically require a substantial degree of user input. For example, in addition to activating an instrument by, for example, closing a foot switch together with another switch (as on a handpiece), a user of known instruments must also select a suitable energizable electrode configuration and threshold values of, inter alia, voltage, current, frequency and waveform (collectively “operating parameters”) corresponding to a desired therapeutic outcome. Many electrosurgical operations require a surgeon or other user to apply a plurality of therapies during a single treatment session, and each therapy often corresponds to a unique combination of energizable electrode configuration and operating parameters.

Often, a desired therapeutic outcome corresponds to a measure of electrical energy applied to a treatment. A suitable measure of electrical energy can be electrical power, though any number of measures of electrical energy corresponding to voltage, current, frequency and waveform are possible. In any event, a suitable electrical power (and other combinations of electrosurgical instrument operating parameters) corresponding to a desired therapeutic outcome can depend heavily on an impedance of a treatment site (sometimes referred to as “tissue impedance”). Tissue impedance can vary among tissue types. For example, skin can have a relatively higher tissue impedance compared to a substantially liquid tissue, like blood.

Accordingly, for a selected combination of energizable electrode configuration and operating parameters (e.g., for a desired therapeutic outcome), electrical energy applied among treatment sites can vary according to each treatment site's tissue composition. Adding to the complexity faced by a surgeon, tissue composition at a given treatment site can vary among patients. Thus, a selected combination of energizable electrode configuration and operating parameters that might be suitable for a given treatment site on one patient can be unsuitable for the given treatment site on another patient, leading to a sub-optimal therapeutic outcome for the other patient.

Considering the state of the art, there remains a need for easy-to-use electrosurgical systems. For example, a need remains for electrosurgical systems configured to adjust a respective electrical output based in part on an actual (or observed) tissue impedance of a treatment site.

A need also remains for electrosurgical systems configured to adjust electrical output to correspond to a desired electrosurgical therapy. For example, a given electrosurgical procedure can comprise a number of different individual electrosurgical therapies applied in a predetermined sequence.

Moreover, tissue impedance of a corresponding treatment site might change as a result of one or more individual electrosurgical therapies applied to the treatment site. Consequently a need remains for an electrosurgical system configured to adjust an electrical output corresponding to a selected electrosurgical therapy (or sequence of therapies), tissue impedance and selected energizable electrode configuration.

### SUMMARY

The innovations disclosed herein overcome many problems in the prior art and address one or more of the aforementioned as well as other needs. In certain instances, the innovations disclosed herein are directed to electrosurgical instruments suitable for use in providing any of a variety of patient therapies (e.g., ablative or non-ablative therapies for providing surgical or aesthetic treatments to patients). Some embodiments of disclosed instruments are reconfigurable, allowing, for example, one instrument design to be suitably configured for a variety of purposes (e.g., to serve a plurality of market segments), rather than having a unique instrument design for each respective purpose.

A given electrosurgical procedure might be most therapeutically effective when administered within a given range of instrument operating parameters (e.g., average, or RMS, power administered to a patient through a given surface area, corresponding to a therapeutically effective energy flux). That said, if an instrument is configured to provide a given voltage potential, as with prior art instruments, and if the instrument's output is not current limited, an actual power (or energy flux) administered to a patient can vary throughout a given treatment according to, for example, impedance variations among different tissues. A treatment having a variable power or energy flux can be less therapeutically effective than a treatment with a substantially constant power or energy flux. Some disclosed instruments are configured to adjust (or limit, or both) one or more operating parameters, for example, an output power or energy flux, responsively to an observed condition, e.g., an observed impedance, external to the instrument.

Some instruments described herein have a modular construction, allowing such instruments to be reconfigured by replacing one or more modules. As another example, some disclosed instruments can be programmed with software or firmware to limit the instrument's available operating parameters (e.g., selected ranges of operating voltage, current, frequency and duty cycle) to those ranges of parameters suitable for a given class of treatments (e.g., non-ablative, aesthetic treatments in one instance, or ablative, surgical treatments in another instance).

Some disclosed instruments can be “self-programming” or “self-configurable” to limit output of selected operating parameters to respective ranges compatible with a given instrument configuration. For example, some disclosed instruments are configured to “recognize” a configuration of an installed handpiece and, at least partially on that basis, adjust available electrical output (e.g., current, voltage, frequency, waveform) to correspond to the configuration of the installed handpiece. In some instances, the handpiece can have a memory (e.g., an EEPROM) programmed with information corresponding to the handpiece configuration, and the “self-configurable” instrument can select available operating parameters to ranges corresponding to the handpiece configuration based on the programmed information (e.g., suitable ranges of each operating parameter for each respective recognizable handpiece configuration can be stored in a look up table).

Some disclosed instruments are configured to adjust available electrical output in correspondence with an observed tissue impedance of a treatment site. For example, a given treatment site can span a variety of tissue types. Consequently, tissue impedance can vary during a given electrosurgical therapy, or among a series of electrosurgical therapies on a treatment site constituting an electro surgical procedure applied to the treatment site. Some disclosed instruments are configured to adjust one or more operating parameters based on an observed (or “instantaneous”) tissue impedance, allowing a user to apply a desired, predetermined quantity and quality of electrical energy to the treatment site.

Disclosed electrosurgical systems are easy to use and generally require less user input than known systems, allowing a user to redirect attention toward therapeutic and clinical matters, and away from adjusting or otherwise “tuning” electrosurgical equipment. By reducing the number of adjustments required by a user, disclosed electrosurgical systems can improve overall patient safety by reducing user errors arising from, for example, improper setting of one or more output adjustments.

The foregoing and other features and advantages will become more apparent from the following detailed description, which proceeds with reference to the accompanying drawings, which form a part hereof, wherein like numerals designate like parts throughout.

### BRIEF DESCRIPTION OF THE DRAWINGS

Unless specified otherwise, the accompanying drawings illustrate aspects of the innovative subject matter described herein.

FIG. 1 shows a sinusoidal waveform having a constant amplitude and a 100% duty cycle.

FIG. 2 shows a composite waveform having less than a 100% duty cycle. The waveform comprises a series of frames, each having a sinusoidal waveform with a constant amplitude followed by no waveform.

FIG. 3 shows another composite waveform having less than a 100% duty cycle. The waveform comprises a series of frames, each having a first sinusoidal waveform with a first constant amplitude followed a second sinusoidal waveform with a second constant amplitude followed by no waveform.

FIG. 4 schematically represents a front panel of an electrosurgical instrument of the type disclosed herein.

FIG. 5 shows a functional block diagram of an electrosurgical instrument of the type disclosed herein.

FIG. 6 shows a schematic representation of a standard 26-pin serial edge connector.

FIG. 7 shows an example of output voltage ripple from a variable output voltage power supply.

FIG. 8 schematically illustrates isolated high-voltage and low-voltage portions of a circuit board, and an opto-electric coupler coupling the portions.

FIG. 9 shows an example graph of power output for an electrosurgical instrument of the type disclosed herein.

FIG. 10 shows a composite output current generated by an electrosurgical instrument of the type disclosed herein.

FIG. 11 shows input signals to a modulator configured to generate a composite output of the type shown in FIG. 10.

FIG. 12 shows another example of input signals to a modulator configured to generate a composite output current for an electrosurgical instrument of the type disclosed herein.

FIG. 13 shows representative output power variation relative to load impedance for each of a variety of operative electrosurgical modes.

FIG. 14 shows another example of a composite output current generated by an electrosurgical instrument of the type disclosed herein.

### DETAILED DESCRIPTION

The following describes various principles related to electrosurgical systems by way of reference to specific examples of electrosurgical instruments (e.g., generators) and related handpieces. As used herein, an “electrosurgical generator” means an instrument configured to supply a suitable voltage potential, and to deliver, when a suitable energizable electrode is operatively coupled to the instrument, a corresponding therapeutic energy to a target site on or in a patient's body. As used herein, a “handpiece” means an instrument configured such that a user can hold it in his hand during use. In some innovative embodiments, a handpiece can comprise an energizable electrode configured to apply a therapeutic energy to a target site on or in a patient's body, or to treat or otherwise manipulate a target site on or in the patient's body.

One or more of the principles can be incorporated in various system configurations to achieve any of a variety of system characteristics. Systems described in relation to particular applications, or uses, are merely examples of systems incorporating the innovative principles disclosed herein and are used to illustrate one or more innovative aspects of the disclosed principles. Accordingly, electrosurgical systems having attributes that are different from those specific examples discussed herein can embody one or more of the innovative principles, and can be used in applications not described herein in detail, for example in ablative surgical applications. Accordingly, such alternative embodiments also fall within the scope of this disclosure.

### Overview

Modular and reconfigurable electrosurgical instruments suitable for use in providing any of a variety of patient therapies (e.g., ablative or non-ablative therapies for providing surgical or aesthetic treatments to patients) are described. In some specific embodiments, an electrosurgical instrument can be configured to adjust its output to correspond to observed tissue impedance. An electrosurgical instrument can be configured provide a given combination of operating parameters suitable for achieving a desired therapeutic outcome based on a user's selection of therapeutic outcome and, for example, configuration of energizable electrode. Some electrosurgical instruments are configured to recognize a configuration or type of energizable electrode automatically.

Some electrosurgical instruments are configured to provide a given class or type of electrosurgical therapies. Some disclosed instruments have a selected combination of instrument modules installed, with each combination of instrument modules being suitable for one or more selected electrosurgical therapies (or procedures). Modular and/or reconfigurable instruments as disclosed herein can improve ease of use and reduce a manufacturer's overall design, manufacturing and distribution costs, by, for example, using a common set of components among a substantial number of (or all) product configurations. Such a modular and/or reconfigurable instrument can be configured according to each of several different uses (or market segments) by selecting a given combination of modules corresponding to a desired use (or market segment).

Some instruments are configured for use primarily (or only) with a monopolar handpiece. Some instruments are configured to operate selectively in a monopolar mode or in a bipolar mode. Disclosed instruments can be configured for use in the surgical market segment, the aesthetic market segment, or both.

A typical instrument configured for use in the surgical market segment delivers a peak output power of about 300 watts RMS (e.g., between about 290 Watts RMS and about 310 Watts RMS) in a CUT mode, and a lower peak power corresponding to each of a COAG, HEMO and FULGURATE mode. A typical instrument configured for use in the aesthetic market segment delivers a peak output power of about 400 watts RMS (e.g., between about 385 Watts RMS and about 415 Watts RMS) in an “aesthetic mode”, about 300 Watts RMS in a surgical CUT mode and a lower peak power corresponding to each of a COAG, HEMO and FULGURATE mode.

A difference between a surgical and an aesthetic version of such a typical instrument relates to a user interface based on, for example, end-user expectations corresponding to the different market segments. For example, market expectations for display size and graphical user input/output characteristics might differ between a surgical market segment and an aesthetic market segment. Nonetheless, the underlying instrument design and modularity can be substantially similar, or identical, between market segments.

As used herein an “electrosurgical mode” means a distinct configuration of an electrosurgical instrument corresponding to a distinct perceived result arising from a given user input. Each distinct configuration can correspond to a physical configuration, a software configuration, a firmware configuration, or a combination thereof. Those of ordinary skill in the art will readily understand that ranges of frequency, duty cycle and amplitude listed herein in connection with any “modes” are merely representative ranges of frequency, duty cycle and amplitude other that those listed expressly herein can be delivered by presently disclosed systems.

In some respects, general functional characteristics of disclosed instruments can best be understood in the context of “modes”. In general, an electrosurgical mode can be considered as a waveform comprising a given frequency, duty cycle, and amplitude (e.g., a maximum power output). Several representative examples of “modes” corresponding to respective therapeutic outcomes follow:
Cut—4 MHz and/or 400 kHz frequency, 100% duty cycle sine wave at 300 watts RMS max power
Blend 1—4 MHz and/or 400 kHz frequency, 50% duty cycle sine wave at 250 watts RMS max power
Coag/Hemo—400 KHz frequency, 25% duty cycle sine wave at 200 watts RMS max power
Fulgurate—400 kHz frequency, very low duty cycle sine wave at 150 watts RMS max power
Aesthetic Mode I—4 MHz, 100% duty cycle sine wave at 300 watts RMS max power
Aesthetic Mode II—4 MHz and 400 kHz mixed frequency 100% duty cycle sine wave at 300 watts RMS max power
Bipolar—4 Mhz, 1.7 MHz, and/or 400 kHz frequency undetermined duty cycle sine wave at 100 watts RMS max power

Each of the modes listed above except the Aesthetic Modes I & II is intended primarily for use in a surgical procedure. Aesthetic Modes I & II are modes intended primarily for use in aesthetic therapies.

The names and/or specifications for the individual modes described herein are merely exemplary in nature and can vary from those presented herein. For example, some instruments described herein are software or firmware programmable. Such instruments allow a given hardware configuration to be tailored to specific market segments, or end uses. For example, a user, a manufacturer, a distributor, a reseller, etc., the ability to define particular waveforms, or modes, in correspondence to a selected therapeutic outcome. As but one example, a given waveform might be relatively more suitable for cutting a first type of tissue than another waveform, whereas the other waveform might be relatively more suitable for cutting a second type of tissue. Instruments as disclosed herein can be programmed with one or the other waveforms to correspond to an end-user's preferred use (e.g., one end-user might be more likely to cut the first type of tissue, so an instrument provided to that end user can be programmed to provide the corresponding more effective waveform when a “cut” function is selected). Those of ordinary skill in the art will readily appreciate that operation of disclosed systems shall not be limited by the representative combinations of frequency, duty cycle and amplitude listed in connection with the foregoing “modes.”

Such market-segment differentiation using the same base hardware can permit a manufacturer to enjoy economies of scale during production, while also being able to supply each distinct market segment with suitable, competitive product.

### Generator Function

An electrosurgical generator produces a high voltage high frequency waveform that, when introduced to patient tissue, produces a desired clinical effect. When electrical energy is introduced into biological tissue it is transformed into heat energy in direct proportion to the impedance of the tissue through which it is traversing. In general the clinical effects of surgical therapies can be summarized as cutting or coagulating tissue or a combination of the two.

Any tissue has the ability to disperse heat energy (generally through conduction). A rate at which a given tissue can disperse energy determines a rate at which a temperature of the tissue increases corresponding to a given rate of energy applied to the tissue. Clinical effects generally correspond to tissue temperature (e.g., upper and lower threshold temperature, and a duration for which the tissue remained within a given temperature range). Thus, in electrosurgery, observed clinical effects generally correspond to a rate at which energy is delivered to a tissue as well as the tissue's ability to disperse the energy.

In cutting, energy is delivered substantially more rapidly than heat can be dispersed within or otherwise removed from the tissue, causing a localized increase in temperature sufficient to vaporize the tissue at the treatment site (e.g., a region in direct contact with a point of energy introduction). A pure cut mode ideally sees no thermal effect outside the vaporization zone. As shown in FIG. 1, a cut waveform 10 is a continuous (100% duty cycle) or near continuous waveform of sufficient power to cause continuous tissue vaporization.

In coagulation, a desired rate of energy flow is lower than in a cutting therapy, achieving a more gradual heating of surrounding tissue. An object of coagulation is to evenly heat tissue to a depth sufficient to achieve hemostasis. If the energy is introduced at a steady state (e.g., with a 100% duty cycle, as described in the cut mode above), the tissue can undesirably exhibit a steep thermal gradient. For this reason energy is usually pulsed—a short burst of high intensity energy (insufficient to achieve vaporization) followed by a “rest period” to allow the concentrated heat to disperse deeper into the tissue.

As shown in FIG. 2, a coagulation waveform 20 can provide repetitive bursts of energy 21a, b, c, followed by intervening periods of low or no energy 22a, b, c. Such an energy flow can be thought of as a series of repeating “frames” 23, each having a predetermined (although not necessarily fixed) duration. Each frame 23 has a corresponding percentage of “on time” 21a, b, c followed by “off time” 22a, b, c to the end of the corresponding frame 23. The percentage of time that energy is supplied during burst 21a, b, c within a given frame is sometimes referred to as a duty cycle. A typical duty cycle can range between less than about 1% to 100%. In some respects, a cut wave form 10 can be considered as a coagulation waveform 20 having a 100% duty cycle.

Insofar as energy carried by a given waveform corresponds to an applied voltage, the effective total energy delivered within a frame 23 is a function of the duty cycle, as described above, and the voltage applied during the “on time” burst 21a, b, c. As such, for a given tissue sample the energy delivered in a 100% duty cycle waveform with a voltage of 100 is the same as the energy delivered by a 50% duty cycle with a voltage 200 although the resulting clinical effects are likely to be quite different between the two waveforms.

In addition, a voltage level required to achieve a given energy transfer further corresponds to an impedance of the tissue at or near the treatment site. In general, a relatively higher tissue impedance requires a corresponding higher voltage to achieve a given energy transfer, since power varies as V²/R, where V is the voltage and R is the impedance.

A tissue impedance, in turn, generally corresponds to a carrier frequency of the electrical current supplying the energy. Typically, a higher relative frequency generally corresponds to a relatively lower tissue impedance, although this correspondence diminishes as frequency increases greatly. Observed tissue impedances can typically vary from less than about 100 Ohms to about 2000 Ohms, such as between about 90 Ohms and about 1900 Ohms.

An electrosurgical generator can produce output waveforms (e.g., carrier frequencies) at a selected one or more of about 400 kHz, about 1.7 MHz, and about 4 MHz. A generator with a selectable output frequency allows a user to adjust the output waveform to achieve a desired clinical result. A higher frequency generally produces a relatively superior cutting action whereas a lower frequency can produce a better coagulation layer with deeper tissue penetration.

Some disclosed electrosurigcal generators are able to produce a waveform with a duty cycle of between less than about 1% and about 100% and sufficient voltage to deliver sufficient power to materials (e.g., tissues) having a range of impedance. In practice, a given generator design can provide a plurality of available duty cycles and one or more upper voltage thresholds, selected combinations of which can provide desired clinical outcomes. Some disclosed generators are configured to compensate for variable tissue impedances, such that a user need not adjust the power, voltage, frequency, duty cycle or current level during operation of the generator as tissues of different impedances are encountered throughout the application of electrical energy to a treatment site.

Some generators are configured to shift (e.g., change, or continuously vary) one or more operating parameters and hence output power quantity and quality within a frame, for example an output voltage level, a carrier frequency, and a carrier waveform, as shown for example in FIG. 3. The example waveform 30 shown in FIG. 3 is merely representative of an output shift within a frame. The waveform 30 has a relatively higher frequency, higher output voltage “burst” 31a, b, c followed by a lower frequency, lower voltage burst 32a, b, c, which in turn are followed by an “off” period 33a, b, c. Although FIG. 3 shows a repetitive sequence of distinct combinations of operating parameters in each frame 34, some disclosed generators can provide a unique sequence of distinct combinations of operating parameters in each frame 34.

In general, a disclosed generator can deliver a plurality of combinations of arbitrary voltage level (e.g., voltage amplitude) and arbitrary carrier frequency within each frame 34. For example, an output burst 35 can be produced from a combination of any selected output voltage with any selected carrier frequency. A plurality of energy bursts 35a, b, c can be combined in a selected sequence within each frame 34 to achieve one or more desired therapeutic outcomes. The selected sequence can repeat or be distinct among the plurality of frames 34 constituting a given therapeutic application of electrical energy.

For example, each burst 35 of high frequency and low frequency energy can vary such that the low frequency burst can precede the high frequency burst and the low frequency burst can have a higher output voltage than the high frequency burst. A duty cycle of each of the low and high frequency portions can vary.

As disclosed in U.S. patent application Ser. No. 11/897,035, an electrosurgical instrument can be configured to generate two carrier frequencies, each having an order of magnitude of about 10⁶ Hertz (e.g., “MHz frequencies”). As one example, a relatively higher carrier frequency can be about 4 MHz and can be used for operation in a CUT and a CUT/COAG mode (e.g., can be suitable for cutting tissue in a first mode and cutting or coagulating tissue in a second mode). A relatively lower carrier frequency can be about 2 MHz and can be used for operation in a HEMO mode and a FULGURATE mode. These four operating modes typically are represented by CUT: full-wave rectified and filtered CW output with maximum average power; CUT/COAG: full-wave rectified but unfiltered, deeply modulated (at 37.5 or 75 Hz rate) envelope output with approximately 70% average to peak power ratio; HEMO: half-wave rectified and unfiltered, deeply modulated (at 37.5 or 75 Hz rate) envelope output with approximately 35% average to peak power ratio; FULGURATE (or Spark-Gap Wave): deeply modulated (3.6 KPPS random rate) with approximately 20% average to peak power ratio. For electrosurgical operations, as opposed to aesthetic operations, selection of a bipolar mode for the electrosurgical instrument disclosed in U.S. patent application Ser. No. 11/897,035 normally corresponds to the HEMO mode.

U.S. patent application Ser. No. 11/897,035 also discloses an electrosurgical instrument configured to generate three carrier frequencies, four operating modes represented by different electrical modulation waveforms, and to combine under control of the user each of the three carrier frequencies with any of the electrical modulation waveforms representing the different operating modes to form a unique set of electrosurgical currents, and to deliver such electrosurgical currents to either of a connected monopolar handpiece or a bipolar handpieces.

As used herein “electrosurgical mode” means a distinct configuration of an electrosurgical instrument corresponding to a distinct perceived result arising from a given user input. Each distinct configuration can correspond to a physical configuration, a software configuration, a firmware configuration, or a combination thereof.

U.S. patent application Ser. No. 11/897,035 discloses that an electrosurgical mode can be established by a user actuating instrument settings on the front panel or by the user actuating buttons on a handpiece or a footswitch connected to the instrument. Mode examples include monopolar and/or bipolar activation, and a user selection of carrier frequency and modulating waveforms representing one of the four operating modes. Typically, the instrument configuration remains at the last user selection until changed by the user. Generally, the modulation frequencies will vary from 0 Hz to 5 KHz. Specifically, in accordance with the invention, the four operating electrosurgical modes are represented by CUT: CW output with maximum (or 100%) average power obtained with full-wave rectified and filtered carrier waveforms; CUT/COAG: approximately 70% average power output achieved with full-wave rectified but unfiltered, deeply modulated (at approximately 100 Hz rate) output waveforms; HEMO: approximately 50% average power output achieved with half-wave rectified and unfiltered, deeply modulated (at approximately 60 Hz rate) output waveforms; FULGURATE (or Spark-Gap Wave): approximately 20% average power output achieved with deeply modulated (3.6 KPPS random rate) output waveforms. The percentages given are with respect to the maximum value.

The various combinations of carrier frequency and modulation combined with the choice of handpiece selected by the user/surgeon produces a remarkable number of active electrosurgical currents with a wide variety of tissue effects. Three different carriers each with four different modulations applicable to tissue via either of two different handpieces provides a total of 12 different electrosurgical currents via the two handpieces and provides, in essence, at selected power levels varied levels of coagulation and cutting. This includes not only the usual high power tissue cutting currents as well as low power bleeder coagulation currents but also more modest tissue effects with controllable lateral heat spread better suited around critical anatomical parts for hemostasis as well as lower frequency currents for application to liquid-heavy surgical procedures. While, generally speaking, the monopolar handpiece is preferred for smooth cutting and combined cutting and coagulation, whereas the bipolar handpiece with its two active ends concentrates the electrosurgical currents between the ends, and is thus preferred for local hemostasis with lower power, many surgical situations may arise where it is preferred that higher power electrosurgical currents are applied with the bipolar handpiece and lower power electrosurgical currents with the monopolar handpiece.

In a preferred embodiment, the first carrier frequency is in the range of about 3.8-4.0 MHz, the second carrier frequency is in the range of about 1.7-2.0 MHz, and the third carrier frequency is in the range of about 400-600 KHz. The preferred values are 4 MHz, 1.71 MHz and 500 KHz.

Preferably, the first, second and third carrier frequencies are derived by division by 2 upon selection from RF carrier generators at double the desired frequencies which simplifies the RF generator selection circuitry.

In accordance with a further aspect of the invention, the instrument is configured so that both a monopolar handpiece and a bipolar handpiece can be used during a surgical procedure, though not at the same time, without having to activate any switches on the instrument.

Since the low and high frequencies can differ by a factor of about 10, the output impedance matching and filtering circuits can require different components and switching techniques.

### Generator Architecture

Generator circuitry can be divided into a plurality of independent modules. For example, each module's circuitry and associated components can be assembled on a corresponding printed circuit board (PCB), sometimes referred to in the art as a “daughter card.” The plurality of daughter cards can be electrically and physically coupled together into an operative configuration, as will be described more fully below.

Such a divided architecture can facilitate development of a first generation product, and can provide a scalable design, e.g., to facilitate design and production of improved generators, generators tailored for specific uses and/or market segments. For example, a selected module can be redesigned or otherwise modified, requiring, in some instances, only the replacement of the corresponding daughter card, rather than all circuitry of the generator.

Each module can have a corresponding interface specification (e.g., form, fit and function) comprising, for example, design requirements, functional (e.g., signal) inputs and outputs, form factor, physical connectivity, etc. Each module's daughter card can be independently designed, subject to defined interface specifications, and each module can be independently tested against design criteria (subject to the defined interface specifications). Typically, an interface specification defines a set of electrical inputs to and outputs from each module.

Consistent with a modular architecture, this section describes substantially only those aspects of the generator common to all modules. Subsequent sections of this disclosure describe aspects of the various generator modules.

As one example, circuitry of a generator configured to deliver output energy corresponding to a desired therapeutic outcome and actual tissue impedance variation can be distributed among a plurality of modules. The following modules are mere examples, as instruments described herein can be partitioned into a variety of different functional blocks (or modules):
Universal Input Power Module (UIPM)—this module can generate a fixed high voltage DC (i.e., direct current) output (e.g., between about 160VDC and about 380VDC) at a maximum power output of 750 watts continuous from a line voltage delivering AC (i.e., alternating current) between about 80 and about 240 VAC (universal input).
Variable Output Voltage Power Supply (VOVPS)—this module can produce two independent, lower voltage outputs (each referred to herein as a “B+ voltage”), each providing a maximum power of about 640 Watts (one at a time) from an input received over, for example, a serial interface from the output of the UIPM.
Waveform Generator Module (WGM)—this module can generate the logic level waveforms (e.g., a “carrier waveform”) to be modulated onto one or both of the B+ voltages.
Output Driver Module (ODM)—this module can modulate the B+ voltages generated by the VOVPS with the waveforms generated by the WGM and can perform impedance matching and output filtering to maintain a selected quality and quantity of output power within a predefined range of one or more set points.
Feedback Sense Module (FSM)—this module can monitor a power delivered to a patient and can adjusts one or more of the B+ voltages accordingly.
Output Selection Module (OSM)—this module can directs the output of the ODM to a selected output connector (e.g., on a front panel of a generator) and can connect return lines, as appropriate, to an output frequency and mode.
User Interface Module (UIM)—this module can control each of the other modules, can monitor their respective functions, can process error messages and user inputs, and can provide an interface for one or more displays, panel switches, indicators and dials.

The User Interface Module can act as a central control center for a particular generator, providing digital (e.g., logic level, serial interface) commands to each of the plurality of modules, as needed to control each module's respective output(s). Each module can return analog (e.g., a proportional frequency output) and/or a digital operational status indicator to the User Interface Module, allowing the UIM to monitor each module's operational state.

A portion of the logic constituting the Feedback Sense Module (FSM) can be integrated into the UIM. Individual modules can provide the real-time data used to assess a performance of the overall energy output and can adjusts each module's respective settings accordingly. As such, the FSM need not be a stand-alone module but rather can have its functionality distributed among various other modules as suitable for a given design.

A front panel of a typical generator can have a conceptual appearance as shown in FIG. 4, although a layout of each of the plurality of interface elements can vary according to preferred design choices. Generally, a selected combination of user interface points can provide a substantially increased functional flexibility compared to prior art generators. For example, a selected combination of controls and displays (e.g., as shown in FIG. 4) can be used for a plurality of generator products, providing, among other advantages, a common look and feel that can, over time, become associated with a particular designer or manufacturer. In general, an overriding benefit to an architecture as described herein is that new generators, capable of delivering different operating parameters, can be developed from a foundational set of hardware by simply modifying a particular module, or, in some instances, simply adjusting software and/or firmware.

In FIG. 4, the RF (radio-frequency) electrical connectors on the front panel show three different configurations. The “Bipolar” connection, as shown, has only two contacts, one for the active output signal and one for the return signal. This connector can be backward compatible with existing bipolar hand-pieces commercially available from the assignee of this application.

The connector labeled “Monopolar” is shown with three larger diameter pins and two additional, smaller diameter pins. The large diameter pin set apart from the rest can provide a monopolar active output signal. The remaining pins can be used for control switches and, in some instances, a low voltage power source for circuitry in the hand-pieces. The three larger diameter contacts can have a footprint compatible with handpieces commercially available from the assignee of this application. For example, the monopolar connector can allow for backward compatibility with existing hand-pieces, while providing sufficient scalability for future hand-piece improvements.

The connector marked “Aesthetic” can also be a monopolar output connector similar to the one labeled “Monopolar” but having a different footprint, preventing its use from anything but specific (e.g., aesthetic) hand-pieces. If the generator has a high power output capability (e.g., about 400 watts), the high power output can be limited to this connector. A standard “Monopolar” (surgical) connector described above can be limited to about a 300 watts continuous output power (or other suitable power).

The extra pins on the monopolar connectors can allow for the development of “intelligent” hand pieces. For example, a handpiece can have an EEPROM or other device configured to indicate to the generator a configuration of the corresponding energizable electrode. By knowing which hand piece is attached to the generator, operating parameters can be set automatically to accommodate any selected characteristics of the hand piece (e.g., limitations of the hand piece). For example, a fine wire used for precision cutting typically will not be able to handle the full 300 watt output capability of the generator without the potential of causing harm to the patient or user and without the potential for the failure of the hand piece device. Therefore, knowing that a fine wire hand piece is attached to the generator can allow the generator to set a maximum output power level automatically without input from a user. In some instances, the user would be unable to set the output power of the generator above the automatically set upper threshold power level.

Another use of an “intelligent” hand piece connection allows for monitoring usage of specific hand pieces. For example, certain hand pieces can have a limited useful life. Usage time can be monitored to ensure that the handpiece is not used beyond its safe operating time limit. Yet another use of an “intelligent” hand piece can be to monitor one or more characteristics of a patient or the environment in which the hand piece is used, either on its own or in conjunction with the generator. As but one example, if the hand piece is used to alter a temperature of a treatment site, the temperature of the surrounding tissue can be measured and reported back to a user either directly through the hand piece or through the generator's user interface. Although not shown in FIG. 4, a bipolar connector also can have additional pins added to allow for expansion into other “smart” electrodes.

Some generators have a footswitch connector on a front side and a back side of the unit for user convenience. Each of the connectors can have the same form factor and can be used interchangeably (though typically not simultaneously). For example, a permanent generator installation might prefer to use a rear connector to keep the footswitch cable away from other cables. A portable installation (e.g., where the generator is set up and torn down after each procedure) typically could use the front connector, as it is more easily accessible to a user. The footswitch connections as well as all the push buttons and non-RF connection points can be logically part of the user interface.

FIG. 5 is a block diagram for an example of a generator according to this disclosure. Interconnections between the various blocks are shown by arrows in an assortment of colors. The bright green arrows depict a flow of high power electrical energy. The red lines depict digital data transfer typically of operating parameters or instructions. In the illustrated block diagram, there are not analog signals transferred from the User Interface Module to the other Modules, though other configurations of generators can incorporate such signals. The dark blue lines show analog and/or digital feedback returning to the User Interface Module.

The light blue line shows a low voltage, low power modulated signal generated by the Waveform Generator Module that can be mirrored in the High Voltage/Full Power Output Waveform. In addition to the analog waveform, there can be at least an overall on/off signal as well as a voltage selector signal. These two digital signals can allow for the control of the Output Driver Module by selecting the appropriate B+ voltage and controlling its overall on/off state.

The brown line shows a low power voltage source for use by the User Interface Module that bypasses the high power switching power supplies so that they can be turned off when not needed. This can help meet certain industry standars (e.g., IEC-60601 requirements published by the International Electrotechnical Commission (IEC)).

The orange line indicates an analog (RMS) feedback from the Output Driver Module of the actual power being put out by the generator. This information can go to the User Interface Module as well, and in some cases need not go to both the Waveform Generator Module and the Variable Output Voltage Power Supply Module, e.g., depending on selected design choices. This signal can be used to allow the Variable Output Voltage Power Supply Module to adjust its output voltage to increase or decrease the power output of the generator as a whole as desired.

The light green arrows indicate a flow of high energy. As a matter of design choice, power deliver can be ground referenced or floating, as preferred based on, for example, noise (interference) levels generated at the various frequencies. In some instances, the power delivery can be floating at relatively lower frequencies and ground referenced at higher frequencies. The difference in referencing can be controlled through the “Output Connector Switch”.

### Logic Level Interconnections:

Logic level interconnections between the User Interface Module and the other Modules generally provide two-way communications. These are depicted as the red and dark blue lines in the diagram in FIG. 5. The red lines show the data flowing from the User interface Module to the various other Modules and the dark blue lines show the status information (analog and digital) flowing back to the User Interface Module.

The orange line, “Output Power Feedback (Voltage and Current)”, is a special case, insofar as it constitutes a part of the Feedback Sense Module (FSM) described above. In some instances (as in the example shown in the drawings), this Module's hardware can be distributed between the Output Driver Module and the User Interface Module. The Output Driver Module can generate a low level analog voltage and current signal proportional to the instantaneous voltage and current being delivered by the Output Driver Module. Alternatively, the Output Driver Module can include hardware to generate a single analog power waveform. The User Interface Module can use these signals to determine the total power delivered within a frame 34 (FIG. 3). The appropriate B+ voltage can then be adjusted up or down to alter the power delivered during one or more subsequent frame(s). This monitoring and adjustment cycle can be repeated during frame, though a hysteresis factor can be built in to allow for time lag of the adjustment.

For the modules that have processors, there is an I2C serial interface that can facilitate communication between the modules and corresponding processors. In the example described in detail herein, the User Interface Module is always the master and the other modules are always the slave, though other generators can be configured differently using known I2C serial interface conventions.

In this example, though, the communications on the I2C lines are initiated by the User Interface Module. The modules attached to the User Module can signal a desire to establish communication with the User Interface Module by pulling their respective interrupt line low. In response, the User Interface Module can query the module generating the interrupt and get a response, as appropriate.

The following Table 1 illustrates an example of a suitable pinout for a 26-pin serial connector:

TABLE 1
Example pinout for UIM (26-pin serial connector)

| | | | | | |
|---|---|---|---|---|---|
| 1 | — | CGND | 2 | PWR | DGND |
| 3 | Input | SCLK | 4 | PWR | DGND |
| 5 | Bi | SDATA | 6 | PWR | DGND |
| 7 | Input | SSEL | 8 | Output | SINT |
| 9 | Input | On/Off | 10 | — | — |
| 11 | — | — | 12 | — | — |
| 13 | — | — | 14 | — | — |
| 15 | — | — | 16 | PWR | DGND |
| 17 | — | — | 18 | PWR | DGND |
| 19 | Output | ANALOG 1 | 20 | PWR | DGND |
| 21 | Output | ANALOG 2 | 22 | PWR | DGND |
| 23 | PWR | +12 VDC | 24 | PWR | +12 VDC |
| 25 | PWR | +12 VDC | 26 | — | CGND |

Direction is relative to the Universal Input Power Module

Electrical interconnections between and among the modules can be made with conventional ribbon cables and dual in-line headers except where power requirements indicate the need for a larger wire gage. In the case of the higher power requirements, the wiring can be point to point with locking connectors keyed to avoid a possibility of making incorrect connections.

For example, a physical connection between the User Interface Module (UIM) and the various other electronic modules can be accomplished by a locking 26 pin two row header, as shown in FIG. 6 (connected by ribbon cable). The header can have a common set of definitions regardless of the Module on which it is implemented, as shown in Table 1, above. The User Interface can have a plurality of such connectors available to facilitate connection to a corresponding plurality of other Modules.

The 26 pin connector can be a through-hole connector with 0.100″ pin spacing such as that shown in FIG. 6. It can have locking tabs and be keyed to prevent connecting the cable in the incorrect orientation. There usually should be sufficient space to the side of the connector for the locking tabs to be released. The locking tabs can be a short version; there need not be strain relief on the ribbon cable connector. All boards can use a right angle connector unless there are other components on the board that are higher than the top of the locking tab.

As noted above, a suitable pin out of the connector is shown in Table 1, though other pin assignments can be determined during design of the Modules. The following describes the example pinout shown in Table 1.

Pins 2, 4, 6, 16, 18, 20, and 22 are connected to DGND to provide a low impedance connection between and among the various daughter cards. Pins 1 and 26 (first and last conductors on a ribbon cable) are connected to CGND to improve shielding of the ribbon cable connectors. On the User Interface Module, the odd pins 7-21 plus the even pins 8-14 can be connected to a Programmable Logic Device (PLD) such as a Field Programmable Gate Array (FPGA) or CPLD (Complex Programmable Logic Device). This allows the functionality of the pins to be defined as needed. As such, the meaning and direction of the signals handled by each pin may differ from that shown in Table 1. Pins 3 & 5 are dedicated to an I2C interface on all modules. In the event that a given module does not implement an I2C interface, pins 3 and 5 are left unconnected (floating).

A signal intended to convey analog information can be converted to a frequency such that the frequency of the signal is proportional to the analog value. With such an approach, the selected frequency range of the signal can be high enough to convey sufficient granularity within “real time”. For example, “real time” is related to the frame time of the generator (assumed to be on the order of about 1 ms). With such a generator, the frequency of the signal should be sufficiently high to assure that within 1 ms enough counts are received to convey an observed analog value with sufficient accuracy. In addition, the frequency of the lowest value (usually 0) should correspond to a non-zero frequency so the system can monitor the activity. For example, if a signal to be conveyed corresponds to a voltage of an output signal, and if the voltage can be as high as about 7000 volts, a suitable frequency range for the signal can be between about 100 KHz and about 800 KHz. In this example, an output frequency of about 100 KHz can correspond to an “analog” output voltage of 0 volts and an output frequency of about 800 KHz can correspond to an output voltage of about 7000 volts, providing a 100 Hz per volt equivalence, providing sufficient granularity to assure that the output voltage can be monitored at an accuracy level of at most a couple of volts. Using such a frequency protocol can allow for a simple passing of analog data between, for example, a high voltage portion and a logic level portion of the board, while maintaining a suitable degree of isolation between the portions.

A logic state of each of the various lines can be determined in such a way that the system is configured to detect if there is an unconnected (e.g., a “broken” or an open) cable. For example, the User Interface Module can provide a weak pull-up on each digital interface line. With such a pull up, a “fault” state for a digital signal would be a logic high. All logic level signals can be a 3.3 VDC for a high state (logic level 1) and 0 VDC for low (logic level 0).

The 12VDC pins can supply 12 volts to each of the modules from the User Interface Module. That is, the User interface can distribute the 12 volts used by all other modules, and can be used within the other modules to power all logic level circuitry. Each module can be responsible for generating the operating voltages it needs from this single 12 volt supply. In the example pinout shown in Table 1, three pins are dedicated to the 12VDC distribution to provide redundancy as well as reduce the source impedance.

### General Design Considerations:

In order to make testing and future expansion of the generator easy, each of the modules can correspond to a separate circuit board, as noted above. Each daughter card can have an identical form factor and can be stacked (allowing for board to board spacing needed by component heights and associated cooling, e.g., airflow, requirements). To the extent possible, commercially available components can be selected, preferably none nearing a known end of production life to allow for low-cost and on-going assembly of the generator.

High voltage circuits can be segregated from the low voltage (logic level) circuits. For example, slots in boards can maintain a suitable creepage and clearance distances. Interaction between the high voltage circuits, including analog signals, can be provided using with opto-isolators (or other suitable isolator) as shown for example in FIG. 8. Opto-isolators (opto-couplers or other suitable isolators) can be specified to have a voltage withstand capability equal to or greater than a selected upper threshold (e.g., a highest expected board voltage). Logic level traces can be routed to avoid the high voltage section of the board and, as applicable, the entire system but particularly the high voltage circuits can conform to IEC 60601-1:2005 3^{rd} edition “General requirements for basic safety and essential performance”, IEC 60601-1-2:2007 “Collateral standard: Electromagnetic compatibility—Requirements and tests”, and IEC 60601-2-2:2009 5^{th} edition “Particular requirements for the basic safety and essential performance of high frequency surgical equipment and high frequency surgical accessories,” or other relevant standard.

The CGND connections on the 26 pin connector can be attached to a chassis ground through the mounting holes on the logic level section of the board. All the mounting holes in the logic level section of the board can be connected together through a trace going around the edge of the board. The mounting holes in the high voltage section do not necessarily need to be connected to CGND and in any case are not typically attached to the CGND trace on the logic level section of the board.

For example, the high voltage portion of the Universal Input Module (UIM) and the Variable Output Voltage Power Supply (VOVPS) need not share a connection to CGND on the high voltage side of the board. They could have a connection to CGND on the low voltage side because they need to have the CGND connection to the 26 pin connector.

The Output Select Module (OSM) does need a connection to CGND on the high voltage side. The generator has the option of having the high voltage output (to the patient) ground referenced or floating. This selection can be made in the OSM via a relay. If the output is ground referenced, it can be accomplished by connecting the patient return line to chassis ground (CGND). CGND can be present on the high voltage side of the board. As per the comments above, the high voltage connection to CGND need not be connected to the low voltage connection to CGND.

In total there are three separate grounds in the example electrosurgical instrument. They are DGND—digital ground, CGND—chassis ground, and PGND—power ground, though not all modules make use of all the grounds in the example design. PGND in particular is only present on the modules that use or generate the high voltage signals. As such, since the User Interface Module and the Waveform Generator Module do not have any direct contact with the high voltage circuits, there are no PGND areas (traces) on those modules in the example.

For modules making use of a processor, the logic level section of the board can be a multi-layer board with a dedicated ground plane. Where there is a multi-layer construction, the top and bottom layers (component and solder sides) can be used as trace layers and the internal layers can be used for power and ground planes. The ground layer can have few or no breaks other than for vias and can be placed on a layer relatively closer to the side of the board on which the processor is placed. Blind vias can be unnecessary.

In addition to the 26 pin header discussed above, it may also be advantageous to place a connector on the board specifically for manufacturing tests. This could, for example, make various voltages and logic and/or timing signals available to an automated tester. This may not be possible on every board given the trace spacing required by the relatively high voltage levels. Alternatively test points can be used as desired. In any case, a single connector does not mix high voltage and low voltage signals in the design example. Separation between the high voltage side of the board and the low voltage side of the board are used to reduce or eliminate signal cross-talk or other EMI.

### Universal Input Power Module (UIPM)

This module takes alternating current (AC) power from the “wall” and generates an intermediate higher direct current (DC) voltage. It can deliver sufficient power to accommodate downstream losses due to conversion and delivery inefficiencies inherent in the system. The output voltage does not need to hold any specific level provided it is compatible with the input requirements of the Variable Output Voltage Power Supply Module, as described more fully below. The example UIPM does not have its own programmable processor.

Example design objectives for the UIPM are as follows:
Accept input power in the range of 88 VAC-264 VAC,
Accommodate an input power frequency range of 50-60 Hz (design for 45-65 Hz),
Limit inrush current to 30 Amps for a maximum of 1.5 second,
Produce a minimum of 750 watts of output power continuously,
Have an 80 PLUS Gold energy rating,
Operate at an ambient temperature between 5 and 45 degrees Celsius at a relative humidity between 25% and 80% (non-condensing),
Be transported & stored at temperatures between −10 and 50 degrees Celsius at a relative humidity between 10% and 95% (non-condensing), and
As applicable—comply with IEC60601-1, IEC60601-1-2, IEC60601-2-2, UL 2601, and CSA 22.2601 standards.

Example UIPM Inputs are as follows:
AC_IN1 & AC_IN2—AC power line
ON_OFF—High Voltage Output Enable Switch

Example UIPM Outputs are as follows:
HV_DC—High Voltage Output (between 160 VDC and 380VDC output)
HV_OUT—Frequency modulated signal proportional to the output voltage (HV_DC).
OUTPUT_GOOD—Digital output to indicate High Output Voltage available—output should be logic high (2.75-3.3VDC) when High Output Voltage is available (output is greater than 90% of the nominal output voltage—greater than 342 VDC based on a nominal output voltage of 380), and low (1K ohm resistor to ground) when output is not available.
18VDC—Low voltage driver supply to supply voltage to drive high voltage MOSFETs (generated on UIPM and made available to other modules as required—located in high voltage section of board.

Other Example UIPM Connections are as follows:
PGND—Power ground is isolated ground specifically for high voltage and high current circuits
12VDC-12VDC supplied by external power supply to power any low voltage level logic circuitry
DGND—Digital logic ground
CGND—Chassis ground (direct connection to earth ground) connecting the mounting holes on the low voltage side of the board to the 26 pin connector

The following summarizes an example UIPM Verification Method:
Universal Input Power Module to be attached to load impedance required to draw 50W, 250W, 500W, and 750W. Each load to be run for a period of 1 hour continuously
Output efficiency measured at 20%, 50%, and 100% power output consistent with 80 PLUS Gold energy efficiency standard
Universal Input Power Module to be located outside an enclosure with no fan for cooling for demonstration (module verification test)

### UIPM Operational Description

The high voltage output of the UIPM can be used to drive the input to the Variable Output Voltage Power Supply Module (VOVPS). There is no set output voltage requirement as long as the output voltage of this module (Universal Input Power Module—UIPM) is compatible with the input requirements of the Variable Output Voltage Power Supply Module, described below, including thresholds for voltage droop and ripple at higher power levels. Generally, as long as the input voltage supplied by the UIPM to the VOVPS is sufficient to allow for proper switching, the assembled electrosurgical system should work even if the output of this module exhibits some “drift”.

FIG. 7 shows an example of voltage ripple that might be seen at the High Voltage Output of the Universal Input Power Module at high current draw. Ripple present on the output of this supply is a relatively unimportant design consideration. Rather, the module needs to supply, in this example, a constant power of no less than about 750 watts at a voltage sufficient to allow for the complete regulation of the Variable Output Voltage Power Supply Module at it max power setting(s). The total output of the unit can meet the output power requirement even if the output voltage is reduced at high current draw. That is, the voltage and current can be measured under full load to determine the output power.

The High Voltage Output (HV_DC) of this supply can be switched on and off. When there is a logic high (2.00-3.3 VDC) at the High Voltage Output Enable Switch (ON_OFF) input, the High Voltage Output can be on. When there is a logic low (less than 0.8 volts) on the ON_OFF input, the High Voltage Output can be deactivated. This input can be pulled low with a 1K resistor so that if there is no input or the input is not connected, the High Voltage Output stays off.

The analog output can be a simple resistor divider network taken from the output of the UIPM. This voltage signal can be converted to a frequency by a voltage to frequency converter such that the analog signal crosses the isolation barrier as a digital frequency modulated signal and presented to the 26 pin connector as the digital signal HV_OUT. This allows the User Interface Module to monitor the output voltage. A pull-up resistor can be provided to assure that the signal is a steady high in the event that there is no voltage present. The time base of the measurement for the UIPM's output voltage need not be particularly fast and the measurement itself need not be particularly accurate.

That is, the User Interface Module can check the output voltage for the purpose of determining that the voltage is within a fairly wide acceptance band of +/−5% over the course of a second or so. As such, the output frequency can be a minimum of about 100 Hz (corresponding to 0 volts output) and can have a high frequency output of about 800 Hz+/−5% at the nominal output voltage of the UIPM (output set value between 160 VDC-380 VDC). The frequency can rise above 800 Hz if the output of the UIPM rises above the nominal set value.

TABLE 2
Example pinout for UIPM
Universal Input Power Module

| # | Direction | Name | # | Direction | Name |
|---|---|---|---|---|---|
| 1 | — | CGND | 2 | PWR | DGND |
| 3 | Input | SCLK | 4 | PWR | DGND |
| 5 | Bi | SDATA | 6 | PWR | DGND |
| 7 | Input | SSEL | 8 | Output | SINT |
| 9 | Input | On/Off | 10 | Output | OUTPUT_GOOD |
| 11 | — | — | 12 | — | — |
| 13 | — | — | 14 | — | — |
| 15 | — | — | 16 | PWR | DGND |
| 17 | — | — | 18 | PWR | DGND |
| 19 | Output | HV_OUT | 20 | PWR | DGND |
| 21 | Output | ANALOG 2 | 22 | PWR | DGND |
| 23 | PWR | +12 VDC | 24 | PWR | +12 VDC |
| 25 | PWR | +12 VDC | 26 | — | CGND |

Direction is relative to the Universal Input Power Module

The digital output indicating High Voltage Output available (OUTPUT_GOOD) can go high when the High Voltage Output reaches 90% of the nominal output voltage level. It can stay high as long as the High Voltage Output is equal to or greater than 90% of the nominal output voltage level. For example, assuming the nominal output voltage level is 380 VDC, OUTPUT_GOOD can go high when the output voltage reaches 342 VDC and stays high as long as the output voltage is at or above 342 VDC. This signal need not be generated from the HV_OUT signal discussed above. It can cross from the high voltage side of the board to the low voltage side of the board with a digital isolation circuit. This approach provides redundancy on the isolation circuit.

The 12 VDC can be supplied by an external power supply through the standard 26 pin connector common to all modules. In the event that the 12VDC is not present, the high voltage output of this module (HV_DC) can remain off (0 VDC), OUTPUT_GOOD can be pulled low, and the analog output HV_OUT can register 0.0 volts.

The pin-out in Table 2 above shows the pins for each of the 26 pins of the standard module connector. This module does not have a serial port and only one of the two analog pins is used, so pins 3, 5, 7, 8, 11-15, 17, and 21 remain unused, as indicated by the cross-hatching. See the section Design Overview for more information.

The low voltage side of the board can be opto-isolated (or isolated using another isolation technique) from the high voltage side of the board. See Design Overview for more information.

As part of the high voltage/low voltage separation, a low voltage source for driving switches (MOSFETs) can be placed on the high voltage side of the board. This power can be generated by the UIPM locally as desired and can be made available to other modules (VOVPS) as desired.

### Variable Output Voltage Power Supply Module (VOVPS)

This module can take the high voltage DC voltage output supplied by the Universal Input Power Module and reduce the voltage as desired to produce the B+ voltages used as inputs to the Output Driver Module. This module can produce two independent voltage outputs each of which can be active simultaneously. However, under normal circumstances only one output will have power drawn from it at a time. As such, although each of the two outputs can supply the full rated power, the total power output requirement for the combined outputs can be set to that of a single output, if desired.

This module can have its own programmable processor for control of the dual regulators. The voltage(s) required to power this processor can be generated internally by this module independently of the two main variable voltage outputs from the 12VDC supplied to the low voltage side of the board on the 26 pin connector.

Example Design Objectives are as follows:
Produce two independent current limited voltage outputs—output voltages for each output range between 10 VDC and approximately 200 VDC (upper limit yet to be determined) and current limits up to 20 amps as shown in the graph below (640 watts output based on an input of 750 watts)
Both output voltages and current limits can be set by software commands—separately for each output
Regulators can be activated and de-activated by software command
Regulators switched by ON_OFF signal from User Interface Module via the 26 pin connector (both software and ON_OFF must signal “on” state for power supply to turn on)
100% duty rating—each output must be able to produce full rated load continuously (one at a time).
Change output voltage at a minimum rate (slew rate) of 10 VDC per milli-second when under 25% or greater load
Operate at an ambient temperature between 5 and 45 degrees Celsius at a relative humidity between 25% and 80% (non-condensing),
Be transported & stored at temperatures from −10 to 50 degrees Celsius at a relative humidity between 10% and 95% (non-condensing), and
As applicable—comply with IEC60601-1, IEC60601-1-2, IEC60601-2-2, UL 2601, CSA 22.2601 standards.

Example Module Inputs are as follows:
Output of Universal Power Supply Module (160 VDC-380 VDC—fixed input voltage but exact value is yet to be determined)
I2C interface for serial commands
Serial command to set the voltage outputs for each of the two outputs
Serial command to set the max current for each of the two outputs
Serial command to activate and de-activate each of the two regulators (not an output switch—this turns the regulator itself on and off)
SSEL—Input from User Interface Module selecting communication with this board. Held high by the VOVPS and pulled low by the UIM to indicate communication.
ON_OFF—Input from the User Interface Module activating the outputs (both at the same time)—Duplicate of the command to activate or de-activate the regulators but works in hardware and on both outputs together
FRAME_SYNC—digital input pulse to indicate the end of a generator output frame—allows synchronization of the current monitoring with the generator output

Example Module Outputs are as follows:
B+1 & B+2—Two independent outputs each individually capable of producing the output as shown in the table above. Only one output will have power being drawn from it at a time (max combined output is 640 watts based on an input power of 750 watts)
B+1_Vout & B+2_Vout—Two analog outputs (digital frequency proportional—100 KHz-1 MHz) proportional to the output voltage of each of the two voltage outputs (+1 & B+2). The analog output should be set such that the nominal maximum voltage output (upper limit of the output voltage is yet to be determined) corresponds to a frequency output of 800 KHz with allowance to have the frequency output rise to as high as 1 MHz in the event the voltage is higher than the maximum possible set value.
B+1_Iout & B+2_Iout—Two analog outputs (digital frequency proportional—100 KHz-1 MHz) proportional to the output current of each of the two voltage outputs (B+1 & B+2). The analog output should be set such that the nominal maximum current output (20 Amps) corresponds to a frequency output of 800 KHz with allowance to have the frequency output rise to as high as 1 MHz in the event the current is higher than the maximum possible set value.
B+1_CL & B+2_CL—This digital output (current limit) is held high when the variable voltage output is in current limit mode. When the variable output voltage is not current limited, the output is held low (1K resistor pull down to ground).
SINT1 & SINT2—Interrupt request to the UIM.

Other Examples of Module Connections are as follows:
Low voltage supply for high voltage side of board (18 VDC)
PGND—Power ground
12VDC—12VDC for use of local digital and analog logic circuits
DGND—Digital logic ground
CGND—Chassis ground

The following summarizes an example of a verification method:
Input to Variable Output Voltage Power Supply provided by Universal Input Power Module or alternate power supply of comparable voltage and current output capability.
Variable Output Voltage Power supply to be set to the minimum possible output voltage (10VDC) and attached to a load resistance sufficient to cause the supply to go into current limit mode. Load gradually resistance reduced to 0 ohms to demonstrate voltage foldback and short circuit protection feature at low voltage condition
Variable Output Voltage Power Supply to be set to the minimum output voltage at which the supply can produce the maximum set current (32 VDC at 20 Amps=640 watts) and attached to a load resistance consistent with the power output and run for a period of 1 hour continuously.
Set current to be continuously reduced to minimum with load resistance as above—voltage to fold back as required to maintain set output current.
Above two steps to be repeated at the maximum output voltage
Variable Output Voltage Power Supply to be located outside an enclosure with no fan for cooling for demonstration

### VOVPS Operational Description:

FIG. 9 shows an example of output power for each of the variable voltage outputs. As can be seen from the chart there is an operating condition during which the supply is in current limited mode and an operating condition during which the supply is in voltage limit mode.

Both supplies are active at the same time. That is, each will have a set voltage and current limit assigned to it and will be started up (under software control). Therefore, under normal operating conditions, at the idle state both outputs are at their set voltages while there is no current being drawn from the either load. The following discussion limits itself to one variable voltage output but the points are equally applicable to the second output (and in the case of more than two power supplies, each additional power supply).

The processor on the Variable Output Voltage Power Supply (VOVPS) is active as long as there are 12VDC present on the 26 pin connector. The high voltage input may or may not be present but should have no effect on the operation of the processor and the communication with the User Interface Module (UIM)

On power-up the set voltage and set current can be indeterminate but the high voltage outputs can be held in their off state (switchers on the switching power supplies should not be operational). In order to have the switchers turned on, the ON_OFF signal on the 26 pin connector (e.g., Pin 9, in the pinout shown in Table 3 can be set to a high and the processor can be given a serial command telling it to switch the switchers on. If either of these is not true, the switchers can stay off, as shown below:

| | ON_OFF - LOW | ON_OFF - HIGH |
|---|---|---|
| PROC SIG? True | OFF | ON |
| PROC SIG? False | OFF | OFF |

To initiate the start-up sequence, 12 volts can be applied to the board. The processor comes up and initiates by setting the output voltage and current to zero and by turning the switchers enable to off. It then waits for a command from the serial port.

A serial port command can be initiated by the User Interface Module. Serial commands are broadcast on the I2C lines. However, the intended target can be selected by having its serial select line (SSEL) pulled low. The serial select line can be pulled to a logic high by a resistor on the VOVPS.

The User Interface Module (UIM) sets a voltage level and a current limit for each of the B+ supplies through serial port commands. The UIM then sets the ON_OFF signal on the 26 pin connector to “on” (high). Lastly, a serial command is given to turn the switcher on each B+ supply on. The turn on commands can be staggered by several milli-seconds to avoid a voltage dip on the input supply. The supplies then come up to their set voltages (assuming the load impedance is high enough to avoid a current limit condition)

TABLE 3
VOVPS Module Pinout Example.
Variable Output Voltage Power Supply Module

| # | Direction | Name | # | Direction | Name |
|---|---|---|---|---|---|
| 1 | — | CGND | 2 | PWR | DGND |
| 3 | Input | SCLK | 4 | PWR | DGND |
| 5 | Bi | SDATA | 6 | PWR | DGND |
| 7 | Input | SSEL | 8 | Output | SINT1 |
| 9 | Input | ON_OFF | 10 | Output | SINT2 |
| 11 | Input | Frame_Sync | 12 | Output | B + 1CurrL |
| 13 | — | — | 14 | Output | B + 2CurrL |
| 15 | FreqOut | B + 1_Iout | 16 | PWR | DGND |
| 17 | FreqOut | B + 2_Iout | 18 | PWR | DGND |
| 19 | FreqOut | B + 1_Vout | 20 | PWR | DGND |
| 21 | FreqOut | B + 2_Vout | 22 | PWR | DGND |
| 23 | PWR | +12 VDC | 24 | PWR | +12 VDC |
| 25 | PWR | +12 VDC | 26 | — | CGND |

Direction is relative to the Variable Output Voltage Power Supply Module

At this point, the current limit digital output (B+#CurrL) can be held low to indicate that the output is in voltage regulation mode. A variable load can be introduced starting at a low level (high impedance load) and gradually increasing (load impedance is reduced). The current limit output stays low as long as the output current generated by the supply is below the set current limit. Once the output current reaches the set limit, further reductions in the load impedance cause the output voltage to start to drop, keeping the current at the set limit. At this time, the current limit output goes high to indicate that the power supply is in current limit mode. The output voltage continues to drop as the impedance is further reduced.

If while in current limit mode the load impedance is decreased such that the output voltage on one of the B+ outputs drops to half the set voltage, the VOVPS can pull the corresponding serial interrupt signal (SINT1 or SINT2—one for each B+ output) low to generate an interrupt to the UIM. The UIM responds by either turning the switcher off or by adjusting the set output voltage and/or current limit. If the B+ voltage drops to one quarter the set voltage, the VOVPS turns the switcher off on its own. The SINT# signal is pulled high again by the VOVPS after the interrupt is serviced.

If the load is removed, the supply can return back to the set voltage limit and, because the output current can be zero, the current limit digital output (B+#CurrL) can go back to the low state to indicate that the system is no longer in current limit mode.

The analog outputs (B+1_Vout & B+2_Vout) can be frequency modulated outputs that produce an output whose frequency is proportional to the actual output voltage. At an output voltage of 0 VDC, the frequency of this signal can be about 100 KHz. At the maximum output set voltage possible, the frequency can rise to about 800 KHz. The frequency can be capable of rising to, for example, about 1 MHz to account for voltage outputs above the upper threshold (e.g., “maximum”) output voltage (error condition) and the frequency can stay at 1 MHz in the event the output voltage rises higher than can be accurately reported. This assures that any voltage error can be adequately monitored. These signals can be isolated between the high voltage and low voltage side of the board with an analog isolation circuit.

The analog outputs (B+1_Iout & B+2_Iout) can be frequency modulated outputs that produce an output whose frequency is proportional to the actual output current. At an output current of 0 amps, the frequency of this signal is 100 KHz. At the maximum output set current possible (20 amps), the frequency can rise to 800 KHz. The frequency can be capable of rising to 1 MHz to account for current outputs above the maximum allowable output current (error condition) and the frequency can stay at 1 MHz in the event the output current rises higher than the can be accurately reported. This assures that any current error can be adequately monitored. These signals can be isolated between the high voltage and low voltage side of the board with an analog isolation circuit.

The FRAME_SYNC input allows the regulator to get average current and voltage data for the purpose of controlling the outputs on a more or less RMS level. The output current requirements can be “choppy” due to the nature of the downstream load. The FRAME_SYNC input allows the regulator to gather voltage and current (mostly current) “information” over the course of the entire frame and determine to determine if the current is above allowable limits.

To achieve a desired slew rate characteristic, the bulk capacitors used on the output side of the regulators can be sufficient to control the ripple inherent in a switch mode power supply. The power supply need to be able to hold up to heavy instantaneous loads without a drop in the output voltage of this power supply module. As such, the bulk output capacitors need not be larger than required to address the ripple issues at the max rated output current. If the output capacitors are too large, the slew rate of the power supply can be negatively impacted.

### Waveform Generator Module

The Waveform Generator Module produces the waveforms used to drive the output in the Output Driver Module. It gets input instructions from the User Interface Module in the waveform to generate and produces this waveform in repeating “frames” whenever it receives an activation command (Output Enable) from the User Interface Module. It is assumed that this module has a programmable processor and that the waveform is generated by some form of a digital waveform synthesizer.

Example Design Objectives are as follows:
Produce a low level analog signal used to drive the waveform requirements of the Output Driver Module
Select and switch between the voltages produced by the Variable Output Voltage Power Supply Module
Build the repeating “frame”
Operate at an ambient temperature between 5 and 45 degrees Celsius at a relative humidity between 25% and 80% (non-condensing),
Be transported & stored at temperatures from −10 to 50 degrees Celsius at a relative humidity between 10% and 95% (non-condensing), and
As applicable—comply with IEC60601-1, IEC60601-1-2, IEC60601-2-2, UL 2601, CSA 22.2601 standards.

Example Module Inputs are as follows:
12 VDC power source to provide power to the programmable processor and digital waveform synthesizer,
Serial command to input waveform parameters, and
Digital logic level input to activate the output—On/Off

Example Module Outputs are as follows:
Low voltage analog waveform—Wave_Out
Digital output to enable the Output Driver Module—B+Enable
Digital output to select the B+ voltage source used by the Output Driver—B+ Select
Digital pulse to the User Interface Module to indicate the start of a “frame”—Frame Sync
Interrupt signal

Other Module Connections examples are as follows:
12VDC—12VDC for use of local digital and analog logic circuits
DGND—Digital logic ground
AGND—Analog logic ground
CGND—Chassis ground

An example of a suitable Verification Method follows:
Waveform Generator set to produce sine wave output at 300 KHz and 4.1 MHz and various (arbitrarily selected at the time of the test) frequencies in between
Duty cycle to be set from 0% to 100% within a 1 mS to 100 mS envelope—each setting (duty cycle and envelope time) to be arbitrarily selected at the time of the test.
Waveform Generator set to produce dual-frequency output with a (random) high frequency component, a (random) low frequency component and an off-time component set within a (random) 1 mS to 100 mS envelope. Duty cycles for each component to be set (randomly) from 0 through 50% (dual output frequency with no off-time).

### WGM Operational Description:

In a standard electrosurgical generator, there are multiple modes that can be accommodated. Each mode can be defined as a frame with an arbitrary waveform (e.g., a simple sine wave with a defined duty cycle as discussed in the Design Overview document). These frames can repeat as long as the digital logic input Output Enable is high. As such, a number of arbitrary waveforms can be defined as discussed in the Design Overview document. These can be selected by the user through the User Interface Module which communicates the user's choice through a serial command to the Waveform Generator Module.

The overall energy output of the electrosurgical generator can be controlled by this Waveform Generator Module (primarily by setting a selected duty cycle) and the B+ voltage supplied by the Variable Output Voltage Module as processed through the Output Driver Module. The Output Driver Module essentially impresses the waveform generated here on the DC B+ voltage produced by the Variable Output Voltage Module.

The low voltage analog waveform typically is a fixed amplitude signal. The amplitude (voltage) of the signal sent to the patient is determined by the B+ voltage used. For example, the signal to be sent to the patient can be as shown in the Generator Output plot shown in FIG. 10. This example shows a waveform with a high frequency high voltage section followed by a section with a lower frequency and lower voltage which in turn is followed by an “off” time.

To achieve this type of signal, the Waveform Generator Module's low voltage analog waveform would look as shown by the Analog Output plot in FIG. 11. Note that the analog waveform coming from the Waveform Generator Module can be the same amplitude. The analog waveform signal can be output twice from the Waveform Generator Module. One copy is the 0-10 VDC waveform put out on the 26 pin connector. The other is sent directly to the Output Driver Module and can have an amplitude suited to the Output Driver Module.

The varying output voltage from the generator to the patient shown in the example in FIG. 11 can be achieved by setting the first B+ voltage to a higher level than the second and then toggling between the two voltages. This is done using the digital output to select the B+ voltage. When the B+ Select signal is low, the first B+ voltage is used in the Output Driver Module, when the B+ Select signal is high, the second B+ voltage is used. This results in a waveform for the B+ Select signal as shown at left. During the time that the output is active the B+ Enable output is held high. When the B+Enable signal goes low, the Analog Output and B+ Select are ignored by the Output Driver Module.

The last signal shown in FIG. 11 is the Frame Sync. This signal can be used by the Feedback and the User Interface Module to monitor and adjust the power levels (B+ voltage) to assure that the desired power is being delivered.

To produce a simple 100% duty cycle cut mode generator output with a single output amplitude, the signal coming from the Waveform Generator Module can produce a series of waveforms such as those shown in FIG. 12. The Analog Output can be a continuous sine wave at the required frequency. The B+ Select signal can be a 50% duty cycle square wave. This divides the power load evenly between the B+ voltage sources both of which can be set to the same voltage. The B+ Enable signal can stay high (on) for the entire time the output is active. The FrameSync signal can produce the same timing pulse as the first example above.

The Waveform Generator Module can be connected to the User Interface Module and the Output Driver Module. The connection(s) to the Output Driver Module can be implemented with discreet wires and the connection to the User Interface Module can be implemented with a standard 26 pin connector and ribbon cable as used by the other modules.

The Waveform Generator Module has no high voltage/high power components and as such can be a low voltage only board similar to the User Interface Module. As such, it does not need to address the isolation issues found on the modules that do have high voltage components. The low voltage analog output of the Waveform Generator Module can bepassed to the low voltage side of the Output Driver Module. The remaining digital signals can be passed to the User Interface Module which then can resend them to the Output Driver Module. The Output Driver Module can implement a desired isolation.

TABLE 4
Example Pinout for WGM connector.
Waveform Generator Module

| # | Direction | Name | # | Direction | Name |
|---|---|---|---|---|---|
| 1 | — | CGND | 2 | PWR | AGND |
| 3 | Output | Wave_Out | 4 | PWR | AGND |
| 5 | Output | ANALOG 2 | 6 | PWR | AGND |
| 7 | Input | On/Off | 8 | PWR | DGND |
| 9 | Bi | SDATA | 10 | PWR | DGND |
| 11 | Input | SCLK | 12 | PWR | DGND |
| 13 | Input | SSEL | 14 | PWR | DGND |
| 15 | Output | SINT | 16 | PWR | DGND |
| 17 | Output | B + Select | 18 | PWR | DGND |
| 19 | Output | B + Enable | 20 | PWR | DGND |
| 21 | Output | FrameSync | 22 | PWR | DGND |
| 23 | PWR | +12 VDC | 24 | PWR | DGND |
| 25 | PWR | +12 VDC | 26 | — | CGND |

Direction is relative to the Waveform Generator Module

An example pinout of a 26 pin connector for a WGM is a shown in Table 4. Wave_Out can be a 0-10 VDC copy of the analog signal sent directly to the Output Driver Module.

On/Off can be an input from the User Interface Module that activates or deactivates all the outputs from this module. In the “Off” state, the waveform generator need not be generating any analog or digital signals.

SDATA, SCLK, SSEL, and SINT are the serial interface connections. The Waveform Generator Module can be a slave on an I2C serial connection. The board can be selected by the User Interface Module by pulling the SSEL pin low. SCLK is the serial interface clock generated by the User Interface Module and SDATA is a bi-directional pin for the serial data.

SINT is used by the Waveform Generator Module to generate an interrupt on the User Interface Module indicating it needs to be serviced. The response from the User Interface Module to an interrupt is to send a query over the serial channel.

### Output Driver Module

The Output Driver Module (ODM) can combine the outputs of the Waveform Generator Module (WGM) and the Variable Output Voltage Power Supply (VOVPS) to produce the high power waveform used to generate a desired therapeutic effect. In brief the ODM takes the low level waveform generated by the WGM and impresses this waveform on the high power (high voltage) DC voltage produced by the VOVPS. This module need not have a processor and can have limited input requirements outside the WGM waveform and the VOVPS B+ voltages. With such a configuration the output of the ODM is largely defined by the WGM waveform and B+ voltage level(s) it is presented.

The frequency range over which the ODM is operational can be limited to the output range of the WGM. As such, the range over which the ODM example can operate is about 400 KHz through about 4.1 MHz. This represents a sizable range in that the highest frequency is more than 10 times the lowest. As such, this module can comprise a plurality of components each designed to address a specific portion of the range. The overall efficiency of this module can also be relatively low. The input power (B+ voltages) source can provide about 640 watts to this module whereas this module can provide a net output of only about 300 watts to the patient.

Example Design Objectives are as follows:
Produce a high power (high voltage) output based on the input waveform and B+ voltage(s)
Select and switch between two B+ voltage sources
Match the input and output impedances based on an output impedance of 200 ohms (bipolar) and 500 ohms (mono-polar)
Provide feedback on the current being delivered
Produce the nominal max output over a load impedance range of 100 to 300 ohms in monopolar mode and 100 to 300 ohms in bipolar mode
Operate at an ambient temperature between 5 and 45 degrees Celsius at a relative humidity between 25% and 80% (non-condensing),
Be transported & stored at temperatures between −10 and 50 degrees Celsius at a relative humidity between 10% and 95% (non-condensing), and
As applicable—comply with IEC60601-1, IEC60601-1-2, IEC60601-2-2, UL 2601, and CSA 22.2601 standards.

Example Module Inputs are as follows:
B+ Voltages—Variable voltage between 0 and 200VDC with power capacity of 640 watts RMS—B+1 and B+2
Analog waveform to be impressed on the selected B+ voltage—min/max frequency of 300 KHZ/4.1 MHz—WGM_WAVE
Digital logic level B+ voltage selection—B+ SELECT
Digital logic level B+ enable selection—B+ ENABLE
Digital logic level module enable—ON_OFF
Digital logic input frame synchronization—FrameSync

Example Module Outputs are as follows:
High voltage analog waveform (to patient)—PAT_PWR
Current flow indicator—0-10 VDC—AI_OUT
Voltage indicator—0-10 VDC—AV_OUT
Patient ground—PAT_GND (either bipolar or monopolar isolated ground)

Other Module Connection examples are as follows:
PGND—Power ground
18VDC—18VDC for use of control circuits on high voltage side of the module
12VDC—12VDC for use of local digital and analog logic circuits
DGND—Digital logic ground
AGND—Analog logic ground
CGND—Chassis ground

An example of a Verification Method follows:
Input power to be provided by Variable Output Voltage Power Supply (above) or equivalent.
Input waveform to be provided by Waveform Generator (above) or equivalent and present a sine wave input at various frequencies including the minimum, maximum, and mid-point frequencies.
Output load impedance set at various levels including the minimum and maximum levels covered by the “Normal Active Load Impedance Range” and the “Rated Load Impedance” (monopolar and bipolar) as well as additional load levels in between.
Output Driver power output level set to various output levels including 10%, 50%, 90%, and 100% of maximum power output capacity.
Each configuration to be run for 4 cycles of 15 minutes each with a down time (off) of 30 seconds between each cycle.
Output Driver to be located outside an enclosure with no fan for cooling for demonstration

ODM Operational Description:

The Output Driver Module can have two major functions. The first is to impress the waveform presented by the Waveform Generator Module onto the high power (relatively higher voltage) current source provided by the Variable Output Voltage Power Supply. The second function can be to provide an impedance match with the load impedance so that the appropriate power can be delivered to the load.

A suitable maximum output power from the generator can be, for example, about 300 watts RMS over a load impedance range of about 200 to about 1500 ohms. An instantaneous power required to meet the 300 watt RMS output can depend on a duty cycle of the waveform packet which in turn can correspond to a selected mode (cut, coag, hemo, etc.) and its corresponding crest factor.

A crest factor can be independent of the output power and for a sine wave is defined as √(2/D) where D is the duty cycle expressed as a decimal (30%=0.30). The overall power delivered by a sine wave with a peak voltage of V, a crest factor of C and load Impedance of Z is defined as (V/C)²/Z. From this it can be seen that the higher the desired crest factor (lower duty cycle), the higher the peak voltage required to produce a given power output into a particular load. Suitable output power specifications are summarized in table below.

TABLE 5
Summary of suitable power specifications

| | | AFS-300 Generator | | |
|---|---|---|---|---|
| | | Low | Medium | High |
| | | Frequency | Frequency | Frequency |
| | | Frequency Range | | |
| | | 300-400 kHz | 1.6M-1.7 Mhz | 4.0-4.4 Mhz |
| | Description | Max Power - Watts | | |
| CUT | 100% Duty Cycle | 300 W @ | — | 300 W |
| | | 3300 Vp-p max | | |
| BLEND 1 | 50% Duty Cycle | 250 W @ | — | 250 W |
| | | 3800 Vp-p max | | |
| COAG/HEMO | 25% Duty Cycle | 200 W @ | 200 W | No |
| | | 4000 Vp-p max | | |
| Fulgurate | Very low Duty Cycle | 150 W @ | | No |
| | | 6500 Vp-p max | | |
| Pelleve I | 100% Duty Cycle (same as | — | — | 300 W |
| | Cut) | | | |
| Pelleve II | (Switching between High | 300 W | — | 300 W |
| | frequency and Low Frequency - | | | |
| | switching rate to be | | | |
| | determined) | | | |
| Bipolar Coag | Duty Cycle | 100 W | 100 W | — |

| | |
|---|---|
| Power Control | Sense output current and adjust voltage to keep Power as constant as possible |
| | from 100 ohms to 3000 ohms |

Based on Table 5, the following table can be extrapolated. In the following Table 6, each value for max current is calculated based on the requirement that the power output stay flat with a load impedance as low as about 100 ohms across all modes.

TABLE 6
Summary of operating modes for an example generator

| | | MAX | | | | |
|---|---|---|---|---|---|---|
| | | POWER | | | | MAX |
| | DUTY | (watts | CREST | V MAX | V MAX | CURRENT |
| MODE | CYCLE | RMS) | FACTOR | (p-p) | (RMS) | (AMPS RMS) |
| CUT | 100% | 300 | 1.41 | 3,300 | 1,167 | 1.73 |
| BLEND 1 | 50% | 250 | 2.00 | 3,800 | 950 | 1.58 |
| COAG/HEMO | 25% | 200 | 2.83 | 4,000 | 707 | 1.41 |
| FULGURATE | 5% | 150 | 6.32 | 6,500 | 514 | 1.22 |
| AESTHETIC I | 100% | 300 | 1.41 | 3,300 | 1,167 | 1.73 |
| AESTHETIC II | 100% | 300 | 1.41 | 3,300 | 1,167 | 1.73 |
| BIPOLAR | 50% | 100 | 2.00 | 3,300 | 825 | 1.00 |
| COAG | | | | | | |

Using the above table, the plot of output power vs. load impedance would look as shown in FIG. 13.

Because the Cut, Aesthetic I and Aesthetic II modes are all listed at 100% duty cycle and with a max power of 300 watts, they result in a single plot line (shown in red in FIG. 13).

The requirement is for the output to remain constant over the impedance range of about 100 ohm to about 3000 ohm. At the highest powers, this might not be possible, but the output can remain close to the set power. Outside the impedance range of about 100 ohm to about 3000 ohm, the output can be lower (and preferably not higher) than the set power. In the event of a short across the output (e.g., a 15 ohm or lower load impedance), the device can go into a skip mode (e.g., turn on briefly to determine if short condition has been resolved and then turn off again if still shorted) or the output can be turned off until a user releases the foot pedal or finger switch.

The Output Driver Module can be operatively coupled to the remaining modules through the 26 pin connector as well as additional discreet connections. The discreet connections can be both on the high and the low voltage side of the board.

On the high voltage side of the board, there can be inputs for the two B+ voltages (B+ 1 & B+ 2) and power ground (PGND). In addition there can be a low voltage input (18VDC) on the high voltage side of the board. The 18VDC input can be used to drive the switches on the high voltage side of the board. The ground side of the 18VDC power can be power ground (PGND). On the high voltage side of the board there can be outputs for patient power (PAT_PWR) and patient ground (PAT_GND). PAT_PWR and PAT_GND can be isolated from all other circuits on the module. A transformer can be used to provide the impedance matching. The secondary side of the transformer provides PAT_PWR and PAT_GND and need not be electrically connected to any circuits on the primary side.

There can be additional discreet connections on the low voltage side of the board. Most significant of these is the waveform input (WGM_WAVE) coming from the waveform generator module (WGM). This can be the same waveform sent by the WGM to the User Interface Module (UIM). It can be provided directly from the WGM (rather than through the UIM) because the UIM need not have the capability to provide an analog output. This input can correspond to another input for analog ground (AGND) to permit this to be a shielded cable.

TABLE 7
Example ODM pinout
Output Driver Module (with Feedback)

| # | Direction | Name | # | Direction | Name |
|---|---|---|---|---|---|
| 1 | — | CGND | 2 | PWR | AGND |
| 3 | Output | AV_OUT | 4 | PWR | AGND |
| 5 | Output | AI_OUT | 6 | PWR | AGND |
| 7 | Input | On/Off | 8 | PWR | DGND |
| 9 | [symbol] | [symbol] | 10 | PWR | DGND |
| 11 | [symbol] | [symbol] | 12 | PWR | DGND |
| 13 | [symbol] | [symbol] | 14 | PWR | DGND |
| 15 | [symbol] | [symbol] | 16 | PWR | DGND |
| 17 | Input | B + Select | 18 | PWR | DGND |
| 19 | Input | B + Enable | 20 | PWR | DGND |
| 21 | Input | FrameSync | 22 | PWR | DGND |
| 23 | PWR | +12 VDC | 24 | PWR | DGND |
| 25 | PWR | +12 VDC | 26 | — | CGND |

Direction is relative to the Output Driver Module

Chassis ground (CGND) can be attached to the 26 pin connector and to each of the mounting screws on the low voltage side of the board.

The remaining connections can be implemented through the 26 pin connector.

AV_OUT is a 0-10 VDC output signal for the voltage being output by this module. This can be the instantaneous voltage output (should look like the output voltage waveform) with 0 volts corresponding to no voltage output and 10 volts corresponding to 125% of an upper voltage threshold (e.g., a maximum voltage the system is capable of producing). That is, if the system is capable of producing about 6500 volts in fulgurate mode, a 10 volt output can corresponds to an output of 8125 volts. (Assuming a 12 bit A/D in the user interface, this can result in a resolution of just under 2 volts.)

AI_OUT is a 0-10 VDC output signal for the current being output by this module. This can be the instantaneous current output (and can look like the output current waveform) with 0 volts corresponding to no current output and 10 volts corresponding to the 125% of the maximum current output the system is capable of producing. That is, if the system can produce 1.73 amps in cut mode, a 10 volt output corresponds to an output of 2.16 amps. (Assuming a 12 bit A/D in the user interface, this results in a resolution of around 0.5 milli-amps.)

On/Off is an input to this module. When this input is low, all high voltage outputs from this module should be inactive. The AV_OUT and AI_OUT output should still be active. When this input is logic high, the module output should respond to the B+ Select and B+ Enable inputs.

B+ Enable turns the output on and off. When B+ Enable is high, the output is active. B+ Select is used to switch between the two B+ voltage sources. When B+ Select is low, B+1 is used as the power source. When B+ Select is high, B+ 2 is selected as the power source. See the specification for the Waveform Generator Module for additional information on B+ Enable and B+ Select.

FrameSync can be provided if desired. It provides a brief pulse every frame, a frame being defined as the time period over which the waveform repeats—the duty cycle is active.

12VDC is used to provide power to all the low voltage circuitry. The module is responsible for generating its own specific low voltage power from this source.

### Feedback Sense Module

The Feedback Sense Module can receive feedback information pertaining to the voltage and current delivered to a patient and can use this information to calculate a power delivered to the patient. As a load impedance increases (e.g., when going from muscle to fat), a corresponding current delivered at a set voltage level decreases. This decreases the net amount of power delivered to the patient. Likewise, as the load impedance decreases (when going from skin to muscle for example) the amount of current delivered at a given voltage increases thereby increasing the net power delivered. In the ideal instance, the power delivered to the patient should stay constant regardless of the load impedance (tissue type).

This calculation made possible by looking at the current and voltage feedback effectively indicates the patient's load impedance. Using this information, the Feedback Sense Module provides input to allow the User Interface Module to adjust the B+ voltage settings for the variable Output Voltage Power Supply to increase or decrease the voltage settings thereby increasing or decreasing the current delivered to the patient and maintaining the net power delivery at the requested level regardless of the load impedance.

### Feedback Sense Module Operational Description:

The generator operates on a variable duty cycle in a variant of a Pulse Width Modulated (PWM) operating method. That is, the voltage (B+1 and/or B+ 2) are set and the waveform is selected. The waveform consists of a frequency (or frequencies) and a duty cycle. This duty cycle is exercised over a repeating time period of approximately 1 ms. This is called the frame as shown in the plot to the left. The challenge for the feedback module is that energy is only delivered during the active portion of the frame but the power calculation needs to address the power as delivered over the entire frame, the average (RMS) power per frame.

The Output Driver Module takes the B+ voltage(s) and the waveform and combines them. As part of this transformation the Output Driver Module isolates the patient from the remainder of the generator and does impedance matching to convert the relatively low impedance of the power supply to the relatively high impedance of the patient. The voltage and current

### Example Module-to-Module Communications Protocol

The example electrosurgical generator described herein is based on a plurality of modules. As noted above, the central module from a control stand-point is the User Interface Module (UIM). A system operator (user) can interact with this module through one or more front panel control buttons, an LCD, LED displays, one or more electrical connectors, other input devices and combinations thereof. The UIM in turn interacts with the remaining peripheral modules to control the generator's behavior, including output.

The user can select a desired operating mode, including a selected output connector, and one or more qualities and quantities of power output (or a desired therapeutic effect) from a series of menus or other input means associated with the UIM. The UIM software can translate these selections into a series of commands to the peripheral modules such that a suitable mode and power corresponding to the desired therapeutic effect are produced by generator and presented to a selected correct output connector.

The UIM can receive information (e.g., feedback concerning an operating state) from one or more of the peripheral modules and can send, if appropriate, “corrective commands” to the modules and can present to the user the operational status of the device.

TABLE 8
Example UIM pinout

| # | Direction | Name | # | Direction | Name |
|---|---|---|---|---|---|
| 1 | — | CGND | 2 | PWR | AGND |
| 3 | Input | ANALOG 1 | 4 | PWR | AGND |
| 5 | Input | ANALOG 2 | 6 | PWR | AGND |
| 7 | Output | On/Off | 8 | PWR | DGND |
| 9 | Bi | SDATA | 10 | PWR | DGND |
| 11 | Output | SCLK | 12 | PWR | DGND |
| 13 | Output | SSEL | 14 | PWR | DGND |
| 15 | Input | SINT | 16 | PWR | DGND |
| 17 | | | 18 | PWR | DGND |
| 19 | | | 20 | PWR | DGND |
| 21 | | | 22 | PWR | DGND |
| 23 | PWR | +12 VDC | 24 | PWR | DGND |
| 25 | PWR | +12 VDC | 26 | — | CGND |

Direction is relative to the User Interface Module

The physical connection between the UIM and the peripheral modules can be implemented primarily through a 26 pin two row header. The pin definitions of this header vary from one peripheral to another although a “generic” configuration as shown in Table 8 can be adopted. All the pins except odd pins between 7 and 21 (inclusive) are common among the peripheral connections, in this example. For the peripheral modules with a processor (the Variable Output Voltage Power Supply (VOVPS) and the Waveform Generator Module (WGM)), the odd pins 9 through 15 implement the communications connection between the peripheral module processor and the UIM processor. An example of a suitable serial protocol is described below.

In the example pinout in Table 8, Pins 3 and 5 are dedicated to analog. A peripheral module can make up to two analog signals available to the UIM. A suitable range of an analog signal is between about 0 to about 10.0 volts. An increased range is suitable to increase the signal to noise ratio. The UIM can reduce the selected 0-10 volt range as needed to accommodate the requirements of the A/D chip inputs. Unused analog lines can be tied either high or low at the module to assure that it does not float; the UIM can have a very weak pull-down (1 MegaOhm) to analog ground (AGND) make sure that unused connectors are stable.

Pin 7 is the On/Off pin. This pin can be common to all modules. This pin can control switching activity on the high voltage side of the module. When the On/Off pin is held low, limited or no activity can occur on the high voltage side of the board. Any clocks, high speed switching (>1 KHz), or oscillators can be inactive. “Standard” AC line voltage signals (50/60 Hz) can be present and need not be turned off. The low voltage side of the board (logic level) can operate as normal. This tends to reduce the amount of electrical noise an inactive module generates. The On/Off pin can be pulled low by the peripheral module with, for example, a 10 KOhm resistor to DGND.

The odd pins 17, 19, and 21 are available to be defined as needed. They can be logic level signals (e.g., not analog) the function of which can be determined and/or changed to suit a particular selected design.

Pins 23 and 25 can provide power to the low voltage side of the peripheral modules. The 12 VDC (and/or DGND and/or AGND) can be isolated from the high voltage side of the board. Each peripheral module can use the 12VDC provided by the UIM to generate the digital and analog voltage required by the peripheral module. A typical single peripheral module can require less than 1 amp at 12VDC. Two pins (23, & 25) can be allocated to reduce power line losses and provide redundancy. The two lines can be joined together at the peripheral module and can be suitably decoupled with a combination of, for example, a ferrite bead (such as Taiyo Yuden FBMH4532HM202-T) and one or more capacitors (0.1 uF and 1 uF ceramic). The ferrite bead and filter capacitors can be placed directly adjacent to the 26 pin connector.

Pins 1 and 26 can be coupled to chassis ground (CGND). Chassis ground can be a single 0.025″ minimum width trace around the edge of the low voltage side of the board on all layers (top, inner, and bottom layers) of the board (guard ring) connecting all the mounting holes with the 26 pin connector. If there is a connection to chassis ground on the high voltage side of the board, it can have its own trace coupled, for example, only to the mounting holes on the high voltage side of the board. Chassis ground on the high voltage side of the module board (if present) typically would not be coupled directly (e.g., through) to chassis ground on the low voltage side of the board.

All even numbered pins can be connected to one of three grounds in this design example. Pins 2, 4, and 6 are connected to analog ground (AGND), pins 8, 10, 12, 14, 16, 18, 20, 22, and 24 are connected to digital ground (DGND), and pin 26 (along with pin 1) is connected to chassis ground (CGND). The low voltage side of every module can be a multi-layer board with at least 4 layers. The layers starting at the top (component side) can be trace, ground, power (and trace if needed), trace. That is, the component side and solder side of the board can be trace layers, and the inner layers can be ground and power. The ground plane should be a solid plane with no traces. It can be a split plane with an AGND section and a DGND section. All analog components and traces can be in the analog section (with the AGND ground plane). The analog and digital ground sections can be connected with a single 0.035″ diameter through-hole wire jumper with the holes on 0.200″ centers. This jumper may or may not be populated depending on the performance of the system as the DGND and AGND signals are already to be connected at the processor on the UIM board.

### Serial Communications Protocol

A suitable serial communications protocol can be implemented with a modified I2C interface. For example, I2C is a master/slave communications protocol in which the UIM can always be set to master. The odd pins 9-15, in this design example, are used to implement this protocol. An example modification can be that there is no address byte sent out by the UIM (master). Instead, the SSEL line can select a desired peripheral device with which the master wishes to communicate.

Pin 9 is SDATA which is the bi-directional data line. Pin 11 is SCLK which is the clock pin which is always driven by the UIM. When a slave (peripheral module) wishes to transmit a byte (word) to the UIM, the UIM knows this by virtue of the command and toggles the clock for the appropriate cycles to complete the communication.

Pin 13 is the SSEL pin. When the UIM wishes to communicate with a peripheral module, the UIM pulls the UIM low and sends a command out on the SDATA line (using SCLK). Peripheral modules ignore any activity on the SDATA and SCLK lines if their SSEL line is held high. Likewise, any response to be sent back from the peripheral module to the UIM can only occur when the SSEL line is held low.

If a peripheral device (slave) wises to have a communication with the UIM (master) it pulls the SINT line low. This line is held low until the master acknowledges the peripheral by pulling the SSEL line for that peripheral low. Communication is then started between the UIM and that peripheral.

It is possible to have interrupted communications. For example, if the UIM sends a partial command to a peripheral module but pulls the SSEL line high part way through the transmission (between bytes, not in the middle of a byte—which would be an error), the peripheral module waits until its SSEL line is pulled low again to complete the communication cycle.

A standard sequence for a communication is as follows:
UIM pulls SSEL line low
UIM starts by placing a single command byte into the I2C serial buffer and the byte is toggled to the peripheral using the SDATA and SCLK pins.
If the command requires extra data bytes from the UIM, these are placed in the serial stream in succession and sent to the peripheral.
If the peripheral is expected to respond with data, the peripheral places the appropriate data in its output register and the UIM clocks the SCLK line to retrieve the data.
The peripheral places the appropriate acknowledge byte into the output register and the UIM clocks the SCLK line to retrieve the acknowledge byte. Every peripheral has its own unique acknowledge byte for each command. This assures that the UIM and the peripheral are communicating correctly.

Odd pins 17 through 19 contain direct digital logic signals specific to the peripheral not associated with the serial (I2C) port.

The design example described herein includes five peripheral modules. The

Universal Input Power Module (UIPM), the Output Driver Module (ODM), and the Output Selector Module (OSM) do not have processors and as such have no serial (I2C) communications. The Variable Output Voltage Power Supply (VOVPS) and the Waveform Generator Module (WGM) have processors. Most of their communication can be accomplished through a serial (I2C) interface. The peripherals that do not have a processor have a narrowly defined function, as described above and summarized below.

The UIPM takes AC power from the “wall” and transforms this into a fixed intermediary DC voltage (200 VDC). This module is either on or off. When it is on, it produces up to 750 watts of DC power at 200 VDC.

The ODM takes the output of the WGM and impresses this upon the B+ voltage produced by the VOVPS. The only variable input required is the selection of the output transformer to be used. The output transformer is chosen by the UIM to assure output at the required voltage based on the mode of operation the user has selected.

The OSM takes the output of the ODM and connects it to the appropriate connector on the front of the AFS-300 generator. This can be either a standard monopolar connector, a dedicated aesthetic connector, or a standard bipolar connector. In addition, the output can be ground referenced or floating depending, in part, on a selected output frequency.

As stated above, the two modules that have processors are the VOVPS and the WGM. Each of these uses serial (I2C) communications to control their functionality.

The VOVPS has the following commands:
1. B+1 voltage level—followed by the data byte of the voltage level 0-255—data value is volts
2. B+ 2 voltage level—followed by the data byte of the voltage level 0-255—data value is volts
3. B+1 current limit—followed by the data byte of the current limit 0-255—data value is current limit*10
4. B+ 2 current limit—followed by the data byte of the current limit 0-255—data value is current limit*10
5. B+ 1 On—no data
6. B+1 Off—no data
7. B+ 2 On—no data
8. B+ 2 Off—no data
9. Status query—data byte returned from VOVPS to be determined

The WGM has the following commands:
1. First segment frequency—followed by the data integer (two bytes—MSB first) of the frequency in KHz
2. First segment duty cycle—followed by the data byte of the duty cycle (1-100)
3. First segment B+ select—followed by the data byte for the B+ voltage selection (‘1’ or ‘2’)
4. Second segment frequency—followed by the data integer (two bytes—MSB first) of the frequency in KHz
5. Second segment duty cycle—followed by the data byte of the duty cycle (1-100)
6. Second segment B+ select—followed by the data byte for the B+ voltage selection (‘1’ or ‘2’)
7. Stop WGM—no data
8. Start WGM—no data
9. Clear WGM frequency table—no data
10. Status query—data byte returned from WGM to be determined
11. Frame duration—followed by the data byte of the frame duration in 0.25 ms increments (1-200)

Each command starts with the ASCII letter of the command as listed above. It is followed (as required) by a single data byte. The acknowledge byte is the ASCII lower case letter of the original command unless the command is not accepted. If the command is not accepted, the acknowledge character is an ‘*’ (asterisk) for both the VOVPS and the WGM. For example, if the voltage level requested is higher than the VOVPS can supply, the VOVPS responds with an ‘*’ for the acknowledge character and ignores the command (stays at the currently set level). Or, if the combined duty cycle for the first and second segment is greater than 100%, the WGM would respond with an ‘*’ and the last duty cycle command would be ignored.

For example, suppose the UIM wishes to set the B+1 output on the VOVPS to a voltage of 100 VDC with a current limit of 2.5 amps and then turn on the B+1 output. The following communications sequence could be followed:
1. UIM pulls the SSEL line for the VOVPS low.
2. UIM sends out the byte ‘A’ to signal that it is setting the B+1 voltage level.
3. UIM sends out the byte 100 (decimal) to signal that the output voltage should be 100 VDC.
4. VOVPS responds with an ‘a’ (lower case A) to signify that it received the command.
5. UIM sends out the byte ‘C’ to signal it is setting the B+1 current limit.
6. UIM sends out the byte 25 (decimal) to set the current limit to 2.5 amps.
7. VOVPS responds with a ‘c’ (lower case C) to signify that it received the command.
8. UIM sends out the byte ‘E’ to turn the B+1 output on.
9. VOVPS responds with a ‘e’ (lower case E) to signify that it received the command.
10. UIM sets the SSEL line for the VOVPS high.

In the case of a request for status from the VOVPS, the following sequence would be followed:
1. UIM pulls the SSEL line for the VOVPS low.
2. UIM sends out the byte T to signal that it is requesting a status update.
3. VOVPS responds with a byte corresponding to the VOVPS status.
4. VOVPS responds with an T (lower case I) to signify that it received the command.
5. UIM sets the SSEL line for the VOVPS high.

The UIM will time out if it expects a response from a module and does not get it in time. The time out is 1 second. The UIM pulls a SSEL line for a module low for a maximum of 1 second. That is, the SSEL line remains low either until the command sequence is complete or until 1 second has elapsed. As soon as a command sequence is complete or if the SSEL line has been low for 1 second, the SSEL line is set high again.

Any time a command aborts (SSEL line is set high prior to the completion of a command sequence) the incomplete command sequence can be aborted and can be started over. In the first example above, the entire command sequence consisted of three commands—one to set the output voltage and another to set the current limit and a final one to turn the output on. The second command (to set the current limit) will only be started after the completion of the first command, that is after the receipt of the ‘a’ (lower case A) acknowledge character from the VOVPS module. Likewise, the third command (to turn the output on) will only be started after the receipt of the acknowledge character for the second command. If either the first or the second command does not send the acknowledge character before a second has elapsed, the SSEL line is set high by the UIM and the incomplete command would be resent if needed.

The VOVPS status byte can be any character starting with the letter ‘A’ (upper case A) up to and including the letter ‘M’ (upper case M). The status bytes for the WGM can be any character starting with the letter ‘N’ (upper case N) up to an including the letter ‘Z’. In this way, there is no overlap between the status bytes of the two modules. The meaning of the status bytes has yet to be determined and will be worked out as development progresses.

For the VOVPS, the voltages and current limits for the B+1 and B+ 2 voltages can be set while the respective output if turned on. That is, the voltages and current limits can be changed dynamically. This allows for the feedback mechanism to increase and decrease the output voltages as needed to control the power being delivered.

For the WGM, the frequencies and duty cycles can only be changed when the WGM is “Stopped”. This is appropriate as the waveform is set for a particular mode and does not change until the selected mode is changed.

In general the WGM command sequence would start with a “STOP” command followed by a “CLEAR frequency table” command. After this the new frequency and duty cycle data would be loaded. If the combined duty cycles of the frequencies entered is less than 100%, the remaining part of the pulse would be off.

### Communications Example

Following is an example of the serial communications to set up an output waveform.

Assume the user interface configures an output with the following characteristics: Blend mode (50% duty cycle) at 150 watts RMS of output power in monopolar mode at 400 KHz. The resulting output waveform could appear as shown in FIG. 14. The described set-up corresponds to a frame, the duration of which is also configurable. The defined frames automatically repeat. In this example, assume the frame duration is selected to be 1 ms.

First, in setting the VOVPS, the system can balance the use of the B+1 and B+ 2 power supplies to divide the heat generated between the two supplies and thereby potentially reduce cooling characteristics (e.g., fan noise). To produce 150 watts RMS at a nominal load impedance of 500 ohms (standard monopolar impedance) at a duty cycle of 50%, the output voltage can be 547 volts RMS, corresponding to a peak voltage of 774 VDC. Assuming a voltage step-up of 6× in the output driver, the B+ drive voltage can therefore be 129 VDC at a peak current of 9.3 amps. Since both B+1 and B+2 are to be used, both can be set to this voltage.

The current limit can be set high enough that it will not be tripped under normal impedance swings but still protect the system from a short condition. A selected lower threshold impedance can be 50 ohms. The feedback system can limit the output to 150 watts down to 50 ohms. At this impedance and 150 watts the B+ voltage would go down to 41 VDC with a peak current of 29.4 amps. However, at a B+ voltage of 129 VDC the peak current based on a 300 watt output would be 18.6 amps. The current limit can protect the device from a short condition. As such, the current limit in this instance can be set to a value of 18.6 amps. The current limit can be reset every time there is a shift in the B+ voltage.

The voltage for B+1 and B+2 can be set to 129 volts and the current limit for each can be 18.6 amps. Setting the command sequence for the VOVPS then results in the following steps:
1. UIM pulls the SSEL line for the VOVPS low.
2. UIM sends out the byte ‘A’ to signal that it is setting the B+1 voltage level.
3. UIM sends out the byte 129 (decimal) to signal that the output voltage should be 129 VDC.
4. VOVPS responds with an ‘a’ (lower case A) to signify that it received the command.
5. UIM sends out the byte ‘C’ to signal it is setting the B+1 current limit.
6. UIM sends out the byte 186 (decimal) to set the current limit to 18.6 amps.
7. VOVPS responds with a ‘c’ (lower case C) to signify that it received the command.
8. UIM sends out the byte ‘E’ to turn the B+1 output on.
9. VOVPS responds with an ‘e’ (lower case E) to signify that it received the command.
10. UIM sends out the byte ‘B’ to signal that it is setting the B+2 voltage level.
11. UIM sends out the byte 129 (decimal) to signal that the output voltage should be 129 VDC.
12. VOVPS responds with a ‘b’ (lower case B) to signify that it received the command.
13. UIM sends out the byte ‘D’ to signal it is setting the B+2 current limit.
14. UIM sends out the byte 186 (decimal) to set the current limit to 18.6 amps.
15. VOVPS responds with a ‘d’ (lower case D) to signify that it received the command.
16. UIM sends out the byte ‘E’ to turn the B+1 output on.
17. VOVPS responds with an ‘e’ (lower case E) to signify that it received the command.
18. UIM sends out the byte ‘G’ to turn the B+2 output on.
19. VOVPS responds with a ‘g’ (lower case F) to signify that it received the command.
20. UIM sets the SSEL line for the VOVPS high.

The ON/OFF line for the VOVPS is set to ‘ON’ if it is not already ‘ON’ to start the power supplies charging up to the requested voltages. If the VOVPS is already up and running (the ON/OFF line is already ‘ON’), the power supply keeps working to the old voltage until the next command to turn on the B+ voltage is received. For example, in the above example, assume that the VOVPS had previously been programmed to output 200VDC on the B+1 and B+2 outputs. The VOVPS would continue to output this voltage until the receipt of the ‘E’ and ‘G’ command (line 16 & 18 in the above example). That is the B+1 output would be maintained at 150VDC until the receipt of the ‘E’ command after which it would work to produce 129 VDC and the B+2 output would be maintained at 150 VDC until the receipt of the ‘F’ command after which it too would work to produce 129 VDC. The VOVPS must be able to shift voltages (up or down—higher or lower) without stopping the output. It must be possible to change the settings on either output (B+1 or B+2) without affecting the other.

To continue with the example, the B+ voltages have now been set in the VOVPS. The next step in configuring the generator is to set the Waveform Generator Module (WGM). A desired waveform in this example is a 50% duty cycle waveform at 400 KHz. The power output has already been addressed by setting the VOVPS.

The command sequence to the WGM is as follows:
1. UIM pulls the SSEL line for the WGM low.
2. UIM sends out the byte ‘X’ to signal that it is setting the frame duration.
3. UIM sends out the byte 0x04 (hex) to set the frame duration to 1 ms.
4. WGM responds with an ‘x’ (lower case X) to signify it received the command.
5. UIM sends out the byte ‘V’ to clear the WGM frequency table.
6. WGM responds with a ‘v’ (upper case V) to signify it received the command.
7. UIM sends out the byte ‘N’ to signal that it is setting the first segment frequency.
8. UIM sends out the byte 0x01 (hex) for the MSB of decimal 400 value.
9. UIM sends out the byte 0x90 (hex) for the LSB of decimal 400 value.
10. WGM responds with an ‘n’ (lower case N) to signify that it received the command.
11. UIM sends out the byte ‘0’ to signal that it is setting the first segment duty cycle.
12. UIM sends out the byte 25 (decimal) to indicate a duty cycle of 25%.
13. WGM responds with an ‘o’ (lower case 0) to signify that it received the command.
14. UIM sends out the byte ‘P’ to signal that it is setting the first segment power source.
15. UIM sends out the byte 1 (decimal) for to indicate the use of B+1.
16. WGM responds with a ‘p’ (lower case P) to signify that it received the command.
17. UIM sends out the byte ‘Q’ to signal that it is setting the second segment frequency.
18. UIM sends out the byte 0x01 (hex) for the MSB of decimal 400 value.
19. UIM sends out the byte 0x90 (hex) for the LSB of decimal 400 value.
20. WGM responds with an ‘q’ (lower case Q) to signify that it received the command.
21. UIM sends out the byte ‘R’ to signal that it is setting the second segment duty cycle.
22. UIM sends out the byte 25 (decimal) to indicate a duty cycle of 25%.
23. WGM responds with an ‘r’ (lower case R) to signify that it received the command.
24. UIM sends out the byte ‘S’ to signal that it is setting the second segment power source.
25. UIM sends out the byte 2 (decimal) for to indicate the use of B+2.
26. WGM responds with an ‘s’ (lower case S) to signify that it received the command.
27. UIM sends out the byte ‘U’ to start the WGM on the new table
28. WGM responds with a ‘u’ (lower case U) to signify that it received the command.

The ON/OFF line for the WGM is set to ‘ON’ if it is not already ‘ON’ to start the waveform output. If the WGM is already up and running (the ON/OFF line is already ‘ON’), the WGM can keep working to the old frequency data until the next command to turn on the WGM is received (command ‘U’). For example, if the WGM is currently outputting a 4 MHz signal at whatever duty cycle and with a frame duration of 20 ms, it continues to do this until the receipt of the ‘U’ command. Once the ‘U’ command is received, the WGM starts the execution of the new frame duration, frequency, duty cycle, and B+ designation at the start of the next frame. The current frame completes with the previously loaded data, in this example a 20 ms frame at a frequency of 4 MHz.

As stated above, the remaining modules can be configured using the dedicated pins on the 26 pin connector. Specifically, the Output Driver Module can use a suitable matching circuit based on the frequency and possibly the output voltage chosen. The Output Selector Module can be set to direct the output to the appropriate connector and to select a ground referenced or floating output.

In the above example, the final output can be as shown (150 watts at 400 KHz and a 50% duty cycle). The power is controlled through the VOVPS and the frequency and duty cycle are controlled through the WGM. The WGM in this case was set with two segments each with a 25% duty cycle. Segment 2 always follows immediately after segment 1. If the frame time is 1 ms, in the example above, the first 0.25 ms would be segment 1 followed immediately by segment 2 which in this case is also 0.25 ms long. The difference between the segments is that the first segment uses B+1 as the power source and the second segment uses B+2. Had the system been set up to use only 1 power source for the entire frame, it could have been defined as a single segment with a 50% duty cycle. If the sum of the first and second segment duty cycle is less than 100%, the remainder of the frame is automatically “OFF”. If the sum of the first and second segment duty cycle is greater than 100%, it is an error condition.

### Other Embodiments

Incorporating the principles disclosed herein, it is possible to design and construct a wide variety of electrosurgical instruments and other systems. Although specific embodiments of electrosurgical instruments have been described, improvements to currently available electrosurgical instruments are contemplated in this disclosure.

The drawings illustrate specific embodiments, but other embodiments may be formed and structural changes may be made without departing from the intended scope of this disclosure. Directions and references (e.g., up, down, top, bottom, left, right, rearward, forward, etc.) may be used to facilitate discussion of the drawings but are not intended to be limiting. For example, certain terms may be used such as “up,” “down,”, “upper,” “lower,” “horizontal,” “vertical,” “left,” “right,” and the like. These terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” surface can become a “lower” surface simply by turning the object over. Nevertheless, it is still the same surface and the object remains the same. As used herein, “and/or” means “and” as well as “and” and “or.”

Accordingly, this detailed description shall not be construed in a limiting sense, and following a review of this disclosure, those of ordinary skill in the art will appreciate the wide variety of electrosurgical systems that can be devised and constructed using the various concepts described herein. Moreover, those of ordinary skill in the art will appreciate that the exemplary embodiments disclosed herein can be adapted to various configurations without departing from the disclosed concepts. Thus, in view of the many possible embodiments to which the disclosed principles can be applied, it should be recognized that the above-described embodiments are only examples and should not be taken as limiting in scope. We therefore reserve the right to claim as our inventions all that come within the scope and spirit of this disclosure, including but not limited to the all that comes within the scope and spirit of the following claims.

We currently claim:

1. An electrosurgical instrument for selectively providing electrosurgical energy to an energizable electrode, the instrument comprising:
an interface configured to issue one or more waveform command signals corresponding to a selected therapeutic result;
waveform generation circuitry configured to deliver in response to the one or more wave form command signals, a corresponding one or more waveforms selected from a first waveform, a second waveform, and a third waveform, each of the first, second and third waveforms being different from the other of the first, second and third waveforms;
a variable output voltage supply configured to supply a selected electrical current to a corresponding output;
output driver circuitry configured to combine at least one of the one or more waveforms with the respective high-voltage electrical current to generate an electrosurgical current suitable for achieving the selected therapeutic result when applied to a treatment site on or in a patient by the energizeable electrode.

2. An electrosurgical instrument according to claim 1, wherein the selected therapeutic result corresponds to a user-input to the interface, an electrical signal representing a selected energizable electrode configuration, an electrical signal representing a load impedance to the electrosurgical energy, or a combination thereof.

3. An electrosurgical instrument according to claim 1, further comprising impedance matching circuitry configured to adjust an output voltage of the variable output voltage supply to maintain a substantially constant power output throughout an observed range of load impedance.

4. An electrosurgical instrument according to claim 1, wherein:
the high-voltage electrical current comprises a first high-voltage electrical current;
the variable output voltage supply is further configured to supply a second high-voltage electrical current;
the output driver is further configured to combine the first high-voltage electrical current with a selected one of the first, second and third waveforms to generate a first current mode and to combine the second high-voltage electrical current with one of the other two waveforms to generate a second current mode.

5. An electrosurgical instrument according to claim 4, wherein the output driver is further configured to combine the first current mode and the second current mode to generate a composite electrosurgical current suitable for achieving the user-selected therapeutic result when applied to a treatment site on or in a patient by the energizeable electrode.

6. An electrosurgical instrument according to claim 1, wherein the energizable electrode constitutes a portion of an electrosurgical handpiece operative to perform an ablative or a non-ablative electrosurgical procedure.

7. An electrosurgical instrument according to claim 1, wherein each of the first, second and third waveforms comprises a sine wave having a frequency selected from the group of frequencies consisting of about 400 kHz, about 1.7 MHz, and about 4 MHz.

8. An electrosurgical instrument according to claim 3, wherein the observed load impedance can vary between about 90 Ohms to about 2000 Ohms.

9. An electrosurgical instrument according to claim 1, wherein a duty cycle of the generated electrosurgical current is between about 5% and about 100%.

10. An electrosurgical instrument according to claim 9, wherein a duty cycle of the generated electrosurgical current is between about 25% and about 75%.

11. An electrosurgical instrument according to claim 5, wherein a duty cycle of the composite electrosurgical current is between about 5% and about 100%.

12. An electrosurgical instrument according to claim 11, wherein a duty cycle of the composite electrosurgical current is between about 25% and about 75%.

13. A modular electrosurgical instrument for selectively providing electrosurgical energy to an energizable electrode, the instrument comprising:
an interface module configured to issue one or more selected waveform command signals;
a waveform generator module configured to deliver in response to the one or more waveform command signals, a corresponding one or more waveforms selected from a first waveform, a second waveform, and a third waveform, each of the first, second and third waveforms being different from the other of the first, second and third waveforms;
a variable output voltage supply module configured to supply a selected electrical current to a corresponding output;
an output driver module configured to combine at least one of the one or more waveforms with the respective selected electrical current to generate an electrosurgical current suitable for achieving the selected therapeutic result when applied to a treatment site on or in a patient by the energizeable electrode, wherein one or more of the interface module, the waveform generator module, the variable output voltage supply module and the output driver module is at least electrically decouplable from the other modules.

14. An electrosurgical instrument according to claim 13, wherein the selected therapeutic result corresponds to a user-input to the interface, an electrical signal representing a selected energizable electrode configuration, an electrical signal representing a load impedance to the electrosurgical energy, or a combination thereof.

15. An electrosurgical instrument according to claim 13, further comprising impedance matching circuitry configured to adjust an output voltage of the variable output voltage supply module to maintain a substantially constant power output throughout an observed range of load impedance.

16. An electrosurgical instrument according to claim 13, wherein:
the selected electrical current comprises a first high-voltage electrical current;
the variable output voltage supply module is further configured to supply a second high-voltage electrical current;
the output driver module is further configured to combine the first high-voltage electrical current with a selected one of the first, second and third waveforms to generate a first current mode and to combine the second high-voltage electrical current with one of the other two waveforms to generate a second current mode.

17. An electrosurgical instrument according to claim 16, wherein the output driver module is further configured to combine the first current mode and the second current mode to generate a composite electrosurgical current suitable for achieving the user-selected therapeutic result when applied to a treatment site on or in a patient by the energizeable electrode.

18. An electrosurgical instrument according to claim 14, wherein the energizable electrode constitutes a portion of an electrosurgical handpiece operative to perform an ablative or a non-ablative electrosurgical procedure.

19. An electrosurgical instrument according to claim 14, wherein each of the first, second and third waveforms comprises a sine wave having a frequency selected from the group of frequencies consisting of about 400 kHz, about 1.7 MHz, and about 4 MHz.

20. An electrosurgical instrument according to claim 15, wherein the observed load impedance can vary between about 90 Ohms to about 2000 Ohms.

21. An electrosurgical instrument according to claim 13, wherein a duty cycle of the generated electrosurgical current is between about 5% and about 100%.

22. An electrosurgical instrument according to claim 21, wherein a duty cycle of the generated electrosurgical current is between about 25% and about 75%.

23. An electrosurgical instrument according to claim 17, wherein a duty cycle of the composite electrosurgical current is between about 5% and about 100%.

24. An electrosurgical instrument according to claim 23, wherein a duty cycle of the composite electrosurgical current is between about 25% and about 75%.

25. An electrosurgical instrument according to claim 13, further comprising a chassis, wherein the interface module, the waveform generator module, the variable output voltage supply module, and the output driver module comprise respective circuit board physically mounted in the chassis and operatively coupled together by one or more cables.
